SEQUENCE LISTING (45 sequences)

SEQ ID NO: 1 - DNA, 41 bp, Artificial Sequence
  Synthetic Primer
  misc_feature (19)..(23): n is a, c, g, t or u
gggagaggguuuaauuacnnnnnguaauuggauccgcaagg

SEQ ID NO: 2 - DNA, 42 bp, Artificial Sequence
  Synthetic Primer
  misc_feature (19)..(24): n is a, c, g, t or u
gggagaggguuuaauuacnnnnnnguaauuggauccgcaagg

SEQ ID NO: 3 - DNA, 45 bp, Artificial Sequence
  Synthetic Primer
  misc_feature (16)..(18): n is a, c, g, t or u
  misc_feature (29)..(30): n is a, c, g, t or u
gggagaggguuuaaunnnuacgaaaguannauuggauccgcaagg

SEQ ID NO: 4 - DNA, 46 bp, Artificial Sequence
  Synthetic Primer
  misc_feature (16)..(18): n is a, c, g, t or u
  misc_feature (29)..(31): n is a, c, g, t or u
gggagaggguuuaaunnnuacgaaaguannnauuggauccgcaagg

SEQ ID NO: 5 - DNA, 48 bp, Artificial Sequence
  Synthetic Primer
  misc_feature (17)..(20): n is a, c, g, t or u
  misc_feature (31)..(33): n is a, c, g, t or u
gggagaggguuuaaaunnnnuacgaaaguannnauuggauccgcaagg

SEQ ID NO: 6 - DNA, 6 bp, Artificial Sequence
  Synthetic Primer
gggaga

SEQ ID NO: 7 - DNA, 24 bp, Artificial Sequence
  Synthetic Primer
ggguuuaauuacguaauuggaucc

SEQ ID NO: 8 - DNA, 10 bp, Artificial Sequence
  Synthetic Primer
cgcgaaagcg

SEQ ID NO: 9 - DNA, 44 bp, Artificial Sequence
  Synthetic Primer
caugacugcagagagugcugauacuggccugucugcagaucaug

SEQ ID NO: 10 - DNA, 22 bp, Artificial Sequence
  Synthetic Primer
gcacatccagtggctaaagcac

SEQ ID NO: 11 - DNA, 21 bp, Artificial Sequence
  Synthetic Primer
agcacctcatctctttgtcgg

SEQ ID NO: 12 - DNA, 19 bp, Artificial Sequence
  Synthetic Primer
agtgaccctcacttcctgt

SEQ ID NO: 13 - DNA, 22 bp, Artificial Sequence
  Synthetic Primer
ggctgaaccaaggatgatgtgg

SEQ ID NO: 14 - DNA, 22 bp, Artificial Sequence
  Synthetic Primer
gtgaaggctgtgtctgagactc

SEQ ID NO: 15 - DNA, 23 bp, Artificial Sequence
  Synthetic Primer
ggaagtggttcaatcacagaccg

SEQ ID NO: 16 - DNA, 21 bp, Artificial Sequence
  Synthetic Primer
atgccacctgactcctcaagt

SEQ ID NO: 17 - DNA, 23 bp, Artificial Sequence
  Synthetic Primer
ccaccaacagaaatgggtcgttc

SEQ ID NO: 18 - DNA, 21 bp, Artificial Sequence
  Synthetic Primer
gagcgttaccaaacctgtctc

SEQ ID NO: 19 - DNA, 23 bp, Artificial Sequence
  Synthetic Primer
agtaaccgcagttctctgtaggt

SEQ ID NO: 20 - DNA, 23 bp, Artificial Sequence
  Synthetic Primer
aggaagaatcctgtgacaagcac

SEQ ID NO: 21 - DNA, 22 bp, Artificial Sequence
  Synthetic Primer
ccgatctctgtggagtagcagt

SEQ ID NO: 22 - DNA, 22 bp, Artificial Sequence
  Synthetic Primer
ctccaggtcatcctgaacaagg

SEQ ID NO: 23 - DNA, 22 bp, Artificial Sequence
  Synthetic Primer
acagcgcgtataggtggttcag

SEQ ID NO: 24 - DNA, 22 bp, Artificial Sequence
  Synthetic Primer
agaggttctgcccgtcttcctt

SEQ ID NO: 25 - DNA, 21 bp, Artificial Sequence
  Synthetic Primer
ttcgtcctcgaactccagctt

SEQ ID NO: 26 - DNA, 23 bp, Artificial Sequence
  Synthetic Primer
gttcttcttcgcctctcacatcc

SEQ ID NO: 27 - DNA, 22 bp, Artificial Sequence
  Synthetic Primer
ctgtgacgagttctaggaccag

SEQ ID NO: 28 - DNA, 22 bp, Artificial Sequence
  Synthetic Primer
gtgtcagagtctcccagtggat

SEQ ID NO: 29 - DNA, 23 bp, Artificial Sequence
  Synthetic Primer
gttctggctgagaactggagtac

SEQ ID NO: 30 - DNA, 22 bp, Artificial Sequence
  Synthetic Primer
tggactacctgcactcggagaa

SEQ ID NO: 31 - DNA, 22 bp, Artificial Sequence
  Synthetic Primer
gtgccgcaaaaggtcttcatgg

SEQ ID NO: 32 - DNA, 23 bp, Artificial Sequence
  Synthetic Primer
gaaacagtgttccatgctagacg

SEQ ID NO: 33 - DNA, 23 bp, Artificial Sequence
  Synthetic Primer
gccgcttatttgattcctgctcc

SEQ ID NO: 34 - DNA, 24 bp, Artificial Sequence
  Synthetic Primer
tgtgatggtgggaatgggtcagaa

SEQ ID NO: 35 - DNA, 24 bp, Artificial Sequence
  Synthetic Primer
tgtggtgccagatcttctccatgt

SEQ ID NO: 36 - DNA, 20 bp, Artificial Sequence
  Synthetic Primer
tggtggctccatcttagccc

SEQ ID NO: 37 - DNA, 18 bp, Artificial Sequence
  Synthetic Primer
cctgcaggtcgactctag

SEQ ID NO: 38 - DNA, 16 bp, Artificial Sequence
  Synthetic Primer
catcgtggtgtcacgc

SEQ ID NO: 39 - DNA, 17 bp, Artificial Sequence
  Synthetic Primer
atcatgtaactcgcctt

SEQ ID NO: 40 - DNA, 16 bp, Artificial Sequence
  Synthetic Primer
  misc_feature (1)..(1): 2′-O-methyl modified deoxynucleotide
  misc_feature (2)..(2): 2′-O-methyl modified deoxynucleotide
  misc_feature (3)..(3): 2′-O-methyl modified deoxynucleotide
  misc_feature (14)..(14): 2′-O-methyl modified deoxynucleotide
  misc_feature (15)..(15): 2′-O-methyl modified deoxynucleotide
  misc_feature (16)..(16): 2′-O-methyl modified deoxynucleotide
acggcactctctgcag

SEQ ID NO: 41 - DNA, 15 bp, Artificial Sequence
  Synthetic Primer
  misc_feature (1)..(1): 2′-O-methyl modified deoxynucleotide
  misc_feature (2)..(2): 2′-O-methyl modified deoxynucleotide
  misc_feature (3)..(3): 2′-O-methyl modified deoxynucleotide
  misc_feature (14)..(14): 2′-O-methyl modified deoxynucleotide
  misc_feature (15)..(15): 2′-O-methyl modified deoxynucleotide
gggaaccggagctga

SEQ ID NO: 42 - DNA, 6 bp, Artificial Sequence
  Synthetic Primer
gcaagg

SEQ ID NO: 43 - DNA, 44 bp, Artificial Sequence
  Synthetic Primer
gaugagugcagagagugcugauacuggccucucugcagaucaug

SEQ ID NO: 44 - DNA, 27 bp, Artificial Sequence
  Synthetic Primer
ucacggcuagcugugaaagguccguga

SEQ ID NO: 45 - DNA, 22 bp, Artificial Sequence
  Synthetic Primer
ugguggcugcaucuuagcccua

(19) United States
(12) Patent Application Publication
(10) Pub. No.: US 2022/0119868 A1
(43) Pub. Date: Apr. 21, 2022

(54) PROFILING RNA-SMALL MOLECULE BINDING SITES WITH OLIGONUCLEOTIDES

(71) Applicant: The Scripps Research Institute, La Jolla, CA (US)

(72) Inventor: Matthew D. Disney, Jupiter, FL (US)

(21) Appl. No.: 17/275,537

(22) PCT Filed: Sep. 11, 2019
(86) PCT No.: PCT/US19/50531

§ 371 Date: Mar. 11, 2021

Related U.S. Application Data
(60) Provisional application No. 62/729,516, filed on Sep. 11, 2018.

(51) Int. Cl. C12Q 1/6811

(52) U.S. Cl. CPC C12Q 1/6811

(57) ABSTRACT

Many RNAs cause disease, however RNA is rarely exploited as a small molecule drug target. Disclosed herein are methods for identifying privileged RNA motif-small molecule interactions to enable the rational design of compounds that modulate RNA biology starting from only sequence. A massive, library-versus-library screen was completed that probed over 50 million binding events between RNA motifs and small molecules. The resulting data provide a rich encyclopedia of small molecule-RNA recognition patterns, defining chemotypes and RNA motifs that confer selective, avid binding. The resulting interaction maps were mined against the entire viral genome of hepatitis C virus (HCV). A small molecule was identified that avidly bound RNA motifs present in the HCV3′ untranslated region and inhibited viral replication while having no effect on host cells. Collectively, this investigation represents the first whole genome pattern recognition between small molecules and RNA folds.

### STATEMENT OF GOVERNMENT SUPPORT

This invention was made with government support under grant number GM97455 awarded by the National Institutes of Health. The government has certain rights in the invention.

### BACKGROUND

RNA has diverse cellular functions and thus is an important target for small molecule chemical probes and lead therapeutics^{1, 2}. The most exploited RNA targets for small molecules are three-dimensionally folded riboswitches³ and ribosomes^{4, 5}. Both are bacterial in origin, and small molecules that target these structures are used clinically as antibiotics and as chemical probes to dissect RNA biology. Small molecule binding occurs via a vast array of complex interactions within the three-dimensional folds of the RNAs, akin to small molecule recognition of proteins. However, the vast majority of cellular RNAs have little defined tertiary structure but extensive secondary structure. Secondary structure is formed via various canonical (base pairs) and non-canonical pairings (internal loops, hairpins, bulges, and multibranch loops). There is a dearth of compounds targeting such RNAs that affect biological function because of limited information of small molecules that bind these RNA folds.

Viral RNAs are a notable class of targets with extensive secondary structure. Studies with antisense oligonucleotides and small molecules have shown that viral RNAs are indeed viable therapeutic targets⁶. As small molecules have broad chemical space and can be derivatized via medicinal chemistry to improve potency and delivery⁷, they are perhaps more medicinally suited for drugging RNAs than oligonucleotides. Currently, the feasibility of drug development for disease-associated or disease-causing RNAs is limited by the lack of data defining small molecule-RNA secondary structure interactions.

Previous work provided a sequence-based lead identification strategy for identifying small molecules targeting RNA dubbed Inforna^{8, 9}. Inforna was enabled by a screening approach dubbed Two-Dimensional Combinatorial Screening (2DCS) in which a library of array-immobilized small molecules is incubated with a library of RNA motifs (secondary structures) commonly found in cellular RNAs^{10, 11}. By sequencing the RNAs that bind to a small molecule that are captured via 2DCS, the most avid interactions are defined, building an encyclopedia of privileged RNA motif-small molecule interactions that can inform drug design¹². Much more data of this type are required to effectively target the myriad of disease-causing RNAs.

### SUMMARY

In various embodiments, provided herein are methods comprising contacting a library of RNA sequences, a complementary antisense oligonucleotide, RNase H, and a small molecule candidate RNA-binding compound and determining cleavage of the RNA sequences in the presence of the compound (“presence cleavage”); and contacting the library of RNA sequences, the complementary antisense oligonucleotide, and RNase H in the absence of the small molecule candidate RNA-binding compound and determining cleavage of the RNA sequences in the absence of the compound (“absence cleavage”); wherein when cleavage is inhibited (e.g., presence cleavage is lower than absence cleavage), the small molecule candidate RNA-binding compound binds to the RNA sequence. The determination of cleavage inhibition can be used to validate the sequence of binding of the small molecule candidate.

For example, the RNA sequence library can comprise a transcriptome, such as a viral, mammalian, or bacterial transcriptome, or can comprise a transcriptome of a virally- or bacterially infected cell, such as a mammalian cell.

In embodiments, the RNA sequence library can comprise one or more of synthetic, semi-synthetic, or natural RNA; or the RNA sequence library can comprise the genome of an RNA virus.

In various embodiments, methods disclosed herein can be carried out in vitro or can be carried out in living cells. The living cells can be virally- or bacterially-infected cells, such that more than a single transcriptome can be the RNA sequence library of a disclosed method.

In various embodiments, methods disclosed herein can be carried out wherein a set of complementary antisense oligonucleotides and a set of small molecule candidate RNA-binding compounds are assayed in a 2-dimensional parallel array.

Features and advantages of the disclosed methods include (i) there is a dearth of methods to identify the binding sites of small molecules within and RNA target in cells; (ii) the method requires no modification of the small molecule; (iii) oligonucleotide design is simple by sequence complementarity to the putative binding site; (iv) multiple oligonucleotides can be tested in parallel with and without the small molecule making the method amenable to multiplexing; (v) method is applicable to any RNA; (vi) the method can be completed in vitro, in cells including host-infected cells, or in vivo.

### BRIEF DESCRIPTION OF THE FIGURES

FIG. 1. The RNA small molecule binders previously identified and chemotypes used to inform construction of an RNA-focused chemical library.

FIG. 2. Properties of small molecules that were screened for RNA-binding capacity in comparison to known drugs in DrugBank. In general, the small molecules have drug-like properties, as evidence by their similar distributions to compounds in Drug Bank.

FIG. 3. Secondary structures of RNA motif libraries A-E and competitor oligonucleotides that mimic regions constant to all library members used in 2DCS (F-J). Note: competitor oligonucleotide J was not used in selections for hairpins. A is SEQ ID NO: 1; B is SEQ ID NO: 2; C is SEQ ID NO: 3; D is SEQ ID NO: 4; E is SEQ ID NO: 5; F is SEQ ID NO: 6; G is SEQ ID NO: 42; H and I are SEQ ID NO: 7; and J is SEQ ID NO: 8, where N indicates any nucleobase.

FIG. 4. Chemical diversity of small molecules that bind RNA and their chemical properties. A, Heat map of Tanimoto coefficients for each hit compound compared to every other hit. B, plots of various drug-like properties for hit compounds compared to the starting small molecule library. In general, the small molecules selected to bind RNA in this study have drug-like properties.

FIG. 5. Structure-activity relationships for structurally related small molecules. A, RNA motifs derived from libraries C-E preferred by compounds 1 and 2. B, RNA motifs derived from libraries C-E discriminated against by compounds 1 and 2. C, 5-nucleotide hairpins (A) preferred by four related compounds, C-E.

FIG. 6: RNA motifs that bind a single compound but do not bind many others, bind many compounds, or do not bind small molecules studied herein.

FIG. 7: 2DCS informs design of small molecule inhibitors of Hepatitis C viral replication. A, schematic of the secondary structures for the HCV genome, with targetable secondary structure motifs in HCV SL I (SEQ ID NO: 43), SL II (SEQ ID NO: 44) and SL III (SEQ ID NO: 45) regions of the HCV 3′ UTR where SL denotes stem loop. B, structures of 7 and 8, and their LOGOS and DiffLogos analyses. C, structures of 9 and 10 and inhibition of HCV replication using SGR-Neo-RLuc-JFH1-2A replicon cells established in Huh-7.5 (human hepatocytes) by 7-10. DMSO and 2′-c-methyladenosine are negative and positive controls, respectively.

FIG. 8: Investigating compound mode of action by profiling the binding site of 8 in vitro and in cells. A, schematic of an antisense oligonucleotide profiling strategy to map compound binding sites in vitro and in cells. B, in vitro analysis of 8's binding site using the approach in panel A. Left, representative gel image of the inhibition of RNase H-mediated cleavage of SL I by 8. Right, secondary structure of a model of WT SLI (SEQ ID NO: 9) and quantification of inhibition of RNase H cleavage by 8. C, cellular analysis of 8's binding site using antisense profiling. Top, addition of 8 inhibits an antisense oligonucleotide from inducing cleavage of the SL I site, as determined by RT-qPCR. Bottom, an ASO complementary to SL I and 8 have a synergistic effect on viral replication. This synergistic effect is less robust with an antisense oligonucleotide that binds 750 nucleotides downstream in a region not known to contribute to replication (“ASO Control”). *, p<0.05; **, p<0.01; ***, p<0.001, as determined by a two-tailed Student t test.

### DETAILED DESCRIPTION

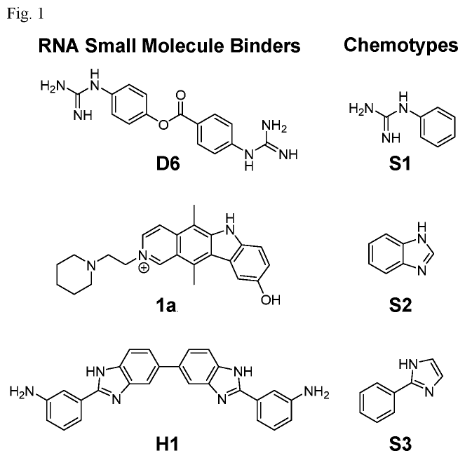

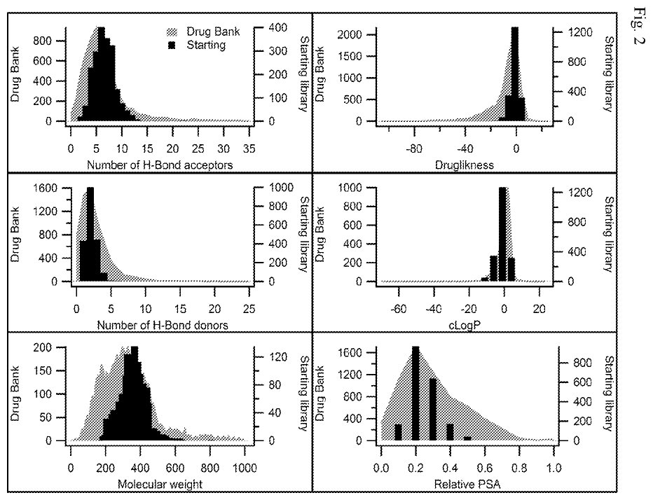

Beyond identifying small molecule RNA binders, one major challenge in the discovery of small molecules directed at RNA is their “drug-likeness”. Aminoglycosides, the most commonly studied small molecules that target RNA, are highly charged, polar compounds and considered very non-drug-like; ironically, they are important drugs used clinically. Herein, a method, termed 2DCS, is used to probe a vast landscape of heterocyclic drug-like small molecule-RNA interactions to identify new chemotypes in small molecules that confer avid binding to RNA and elucidate their RNA motif binding preferences (FIG. 1). Various RNA motif libraries were probed for binding to microarray-immobilized small molecules, totaling over 52 million possible interactions, one of the largest screening campaigns known to date. These studies defined a chemical code for recognizing RNAs with small molecules. For example, it was found that aminopyrimidine chemotypes, a drug-like scaffold found in many clinically tested compounds including kinase inhibitors, bind avidly to RNA motifs. Importantly, the small molecules that bind RNA are indeed drug-like as their properties are similar to those found in FDA-approved drugs, increasing the potential of these compounds to target RNAs in vivo. Thus, decoding RNA with small molecules can be achieved with small molecules that have drug-like properties.

Small Molecule Libraries. Over 30,000 compounds from The Scripps Research Institute (TSRI) and the National Cancer Institute (NCI) small molecule libraries were inspected to identify members that contain an amine for site-specific conjugation onto aldehyde-functionalized microarrays. To reduce the number of compounds to a manageable number for screening, three small molecules known to bind toxic RNAs in cellulis and improve disease-associated defects (D6, 1a, and H1¹⁸⁻²¹; FIG. 1) were used as model compounds for refinement in two ways. First, the three small molecules were subjected to chemoinformatic analysis, generating three scaffolds or sub-structures, phenylguanidine (S1), benzimidazole (S2), and 2-phenyl-1H-imidazole (S3), that likely impart RNA binding affinity²². Compounds that contain at least one of these scaffolds were then selected for further study. In the second refinement, D6, 1a, and H1 were used directly as query molecules in a chemical similarity search of TSRI's library; chemically similar structures (Tanimoto²³>0.25) were carried forward for screening.

The average Tanimoto scores of selected compounds were 0.28±0.05, 0.37±0.08, and 0.37±0.13 as compared to D6, 1a, and H1, respectively. The two refinements afforded 1,987 compounds that were commercially available. Notably, both the library of 1,987 compounds and the >30,000 from which they were selected are chemically diverse, as determined by using a Tanimoto analysis²³. Further, the starting library contains both N- and O-containing heterocycles and functional groups (25% of the compounds contain an oxygen as part of a heterocycle or alcohol; 55% of the compounds have at least one oxygen as an aldehyde, ketone, ester, or amide).

Further, the 1,987 small molecules screened were verified to have drug-like properties by comparing them to the compounds in DrugBank, a publicly available repository containing the properties of FDA-approved therapeutics²⁴⁻²⁶. The compounds were scored for lipophilicity²⁷⁻³⁰ using Log P and Log D values³¹⁻³³. Both values report partition coefficients for the ratio of unionized species in n-butanol to unionized species in water; Log D utilizes an additional algorithm to account for ionized species. The distribution pattern of the Log P and Log D values correlates well between the chemical library and DrugBank compounds with the most compounds having values between 1 and 4. Additionally, the small molecules studied herein and FDA-approved drugs followed similar distribution trends for diversity, molecular weight, lipophilicity, and rotatable bonds (FIG. 2).

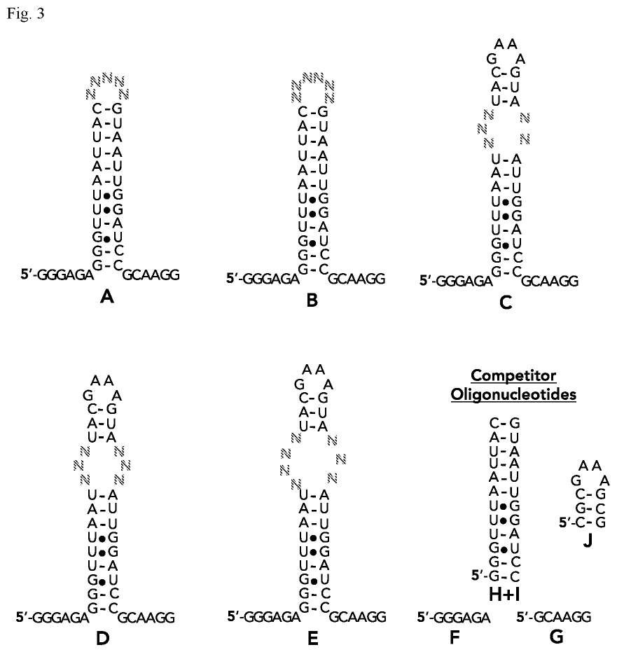

Identification of RNA-binding Small Molecules by 2DCS. To test the compounds for their ability to bind RNA, they were conjugated to a microarray surface that displays aldehydes and incubated with radioactively labeled RNA motif libraries. The secondary structures displayed by the RNA libraries (FIG. 3) were varied to define compound preferences, including hairpin libraries with five (1,024 unique members; A) or six randomized nucleotides (4,096 unique members; B); 3×2 nucleotide asymmetric internal loops (1,024 unique members; C), 3×3 nucleotide symmetric internal loops (4,096 unique members; D), and 4×3 nucleotide asymmetric internal loops (16,384 unique members; E). We have previously validated the secondary structures formed by members of RNA libraries by enzymatic mapping^{8, 12, 34}. These discrete RNA motifs were selected because they are present in cellular RNAs. Due to the library-versus-library nature of these studies, they represent the largest screens completed to date, probing >52,000,000 interactions. Of the 1,987 compounds probed, 239 bound RNA (12.0%).

To remove compounds that non-selectively bind RNA motifs, the 239 small molecules with affinity for RNA were probed for binding to the RNA libraries in the presence of 1,000-fold excess bulk tRNA, affording 91 unique compounds (4.6%; Table 3). A final screen was then completed in which the 91 array-immobilized compounds were incubated with all five RNA motif libraries separately in the presence of oligonucleotide competitors F-J, d(AT)₁₁ and d(GC)₁₁. Oligonucleotides F-J mimic regions common to all library members, restricting binding to the randomized regions. (Note: oligonucleotide J was not used for hairpin selections.) After rigorous washing to remove unbound RNAs, bound RNAs were harvested and identified by RNA-seq. By completing selections under conditions of high oligonucleotides stringency (by use of excess competitor oligonucleotides), these studies identified small molecules that bound RNA motifs avidly. A challenge in the small molecule RNA-targeting area has been the development and identification of selective interactions between small molecules and RNAs. In myriad studies, 2DCS has defined selective RNA motif-small molecule interactions with varied affinities^{11, 12, 35, 36}.

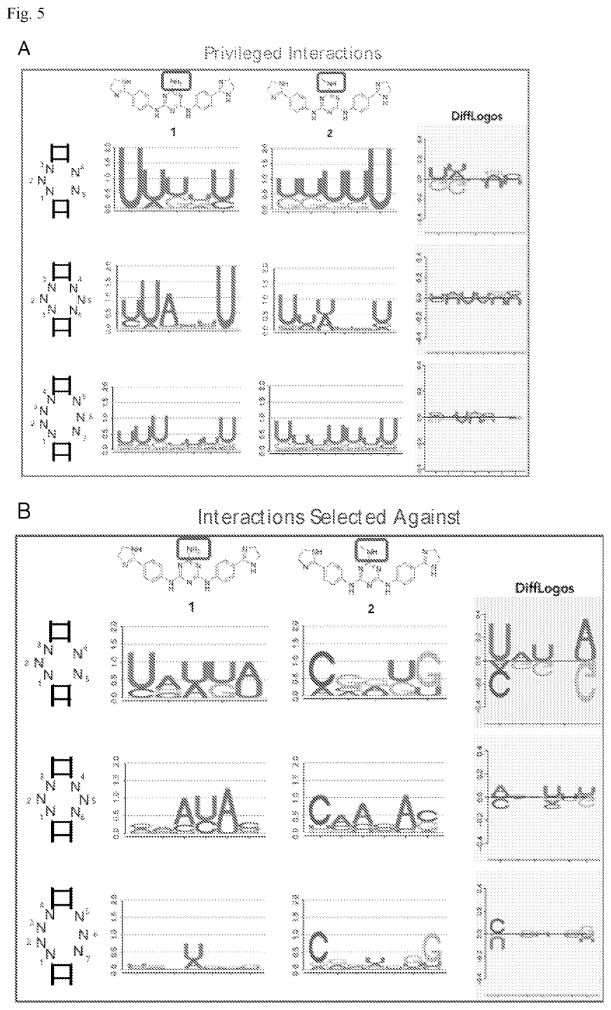

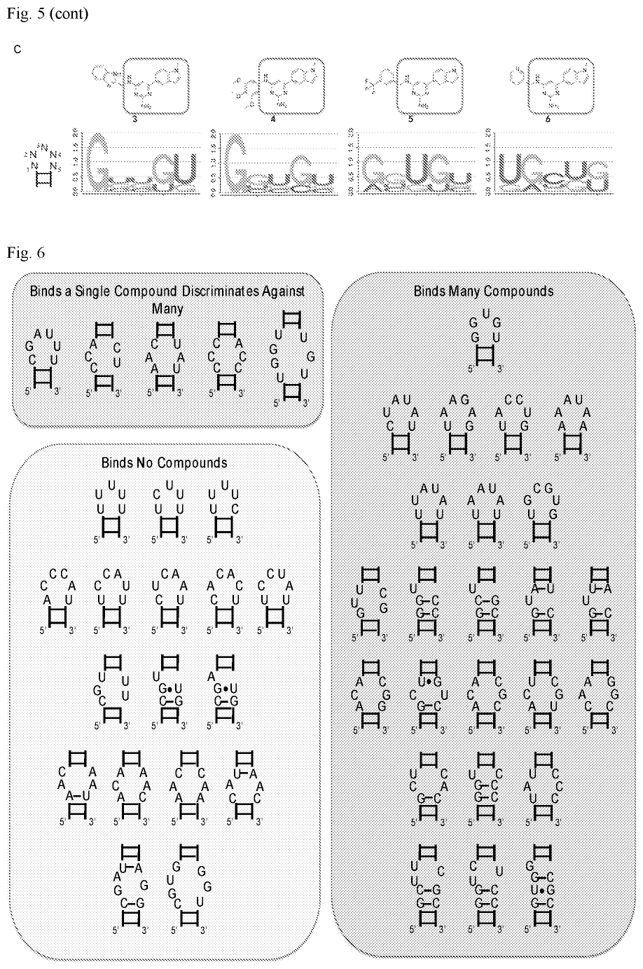

Identification of Privileged RNA Space by RNA-Seq. Using RNA-seq, a large sequencing dataset was obtained for each pool of RNAs that were specifically bound to each small molecule. RNA libraries A, B, C, D, and E had at least 12.1-fold (average: 133.1±111.8), 6.7-fold (average: 80.8±78.7), 9.9-fold (average: 103.5±110.6), 7.2-fold (average: 75.7±65.6), and 6.0-fold (average: 36.3±31.6) coverage for each small molecule selection, respectively, as compared to the number of unique sequences within the corresponding RNA library. It was previously shown that at least 6-fold coverage is required to generate binding landscape maps¹². RNA-seq data were then analyzed by High Throughput Structure-Activity Relationships Through Sequencing (HiT-StARTS)¹² to identify the privileged RNA motifs that bind each small molecule. Briefly, the frequency of occurrence of a selected RNA was compared to the frequency of occurrence of the same RNA from RNA-seq analysis of the starting RNA library to account for biases arising during transcription and RT-PCR. This pooled population comparison affords the parameter Z_{obs}, a metric of statistical confidence. A large, positive Z_{obs} indicates a strong preference for binding the motif while a negative Z_{obs} indicates a strong preference against binding the motif. A Fitness Score is assigned by normalizing the Z_{obs} values to the most statistically significant small molecule-RNA interaction to 100. The RNA binding landscape of a series of substituted benzimidazoles that do not bind DNA was previously studied. From these studies, it was determined that a Z_{obs}>8 defined selective interactions; that is high affinity binding was observed to selected RNA motifs with Z_{obs}>8¹².
Structure-Activity Relationships (SAR). Next, the most privileged RNA motif binders and the most discriminated against RNA motifs (non-binders) for each compound selection was analyzed by generating LOGOS from the highest and lowest 0.5% of Z_{obs} scores³⁷. By comparing the LOGOS for related compounds, or DiffLogos, SAR can be defined. Indeed, various hit compounds differ by a single functional group, including compounds A and B (FIG. 5). Both compounds bind RNA libraries C, D, and E under conditions of high oligonucleotide stringency but do not bind RNA library A or B. Interestingly, both compounds prefer U-rich internal loops regardless of loop size or symmetry (3×2, 3×3, or 4×3) (FIG. 5A). In the case of 2, LOGOS analysis reveals the potential preference for GU closing base pairs (if nucleotide 1 paired with 5; nucleotide 3 with nucleotides 4 or 5, etc.). In contrast, the RNA motifs most hindered from binding compounds 1 and 2 (discriminated against) are quite different (FIG. 5B).

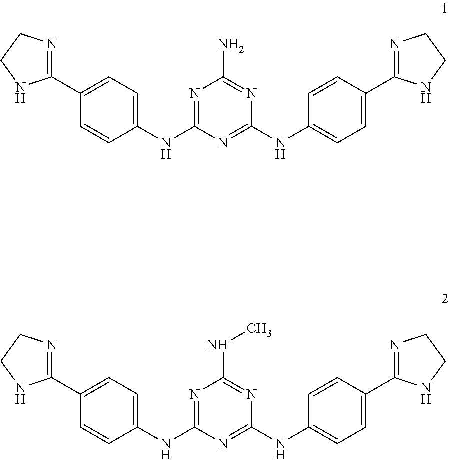 [SMILES: Nc1nc(Nc2ccc(C3=NCCN3)cc2)nc(Nc2ccc(C3=NCCN3)cc2)n1 ; CNc1nc(Nc2ccc(C3=NCCN3)cc2)nc(Nc2ccc(C3=NCCN3)cc2)n1]

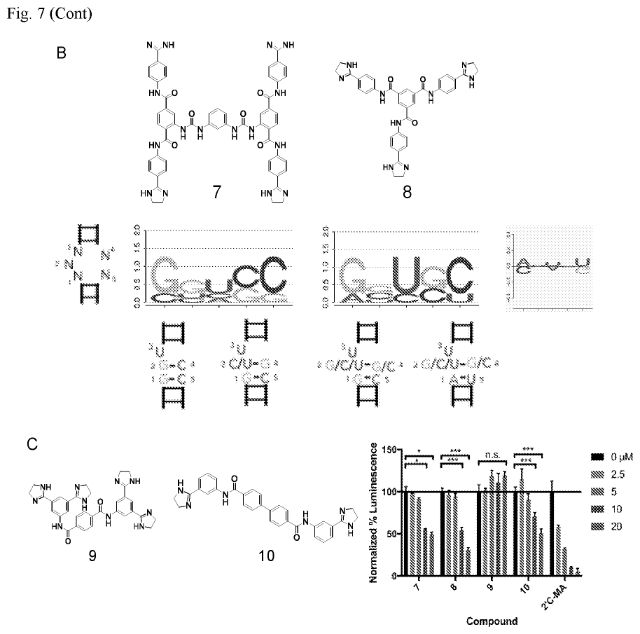

Four compounds that share an indolylpyrimidine-2,4-diamine core bound members of A (5-nucleotide hairpin library). LOGOS analysis shows both similarities and differences, driven by substitution of the diamine (FIG. 5C). For example, compound 3 and 4 have strong preference for G in position 1; 3 and 5 prefers G>U>>A or C in position 4 while 6 prefers G≈U>>A or C, and 4 prefers G>C>>A or U. Additional examples as well as LOGOS and DiffLogos analysis for all compounds are provided in FIG. 7A.

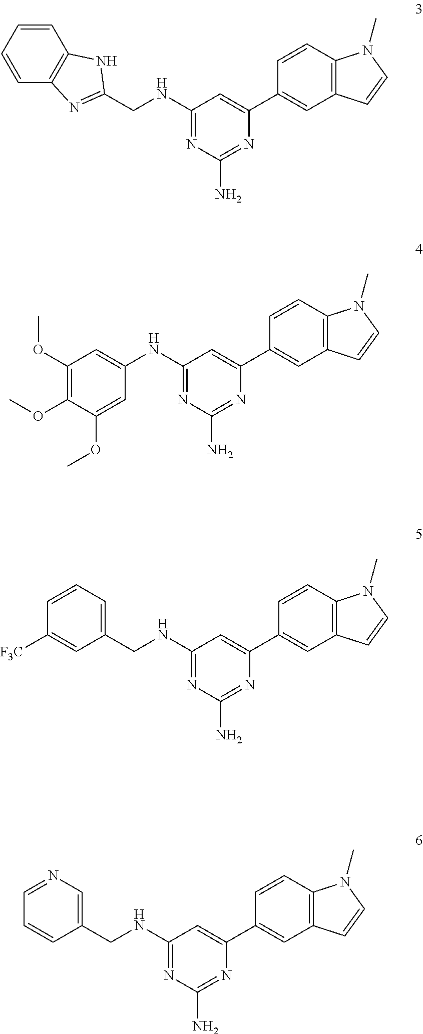 [SMILES: Cn1ccc2cc(-c3cc(NCc4nc5ccccc5[nH]4)nc(N)n3)ccc21 ; COc1cc(Nc2cc(-c3ccc4c(ccn4C)c3)nc(N)n2)cc(OC)c1OC ; Cn1ccc2cc(-c3cc(NCc4cccc(C(F)(F)F)c4)nc(N)n3)ccc21 ; Cn1ccc2cc(-c3cc(NCc4cccnc4)nc(N)n3)ccc21]

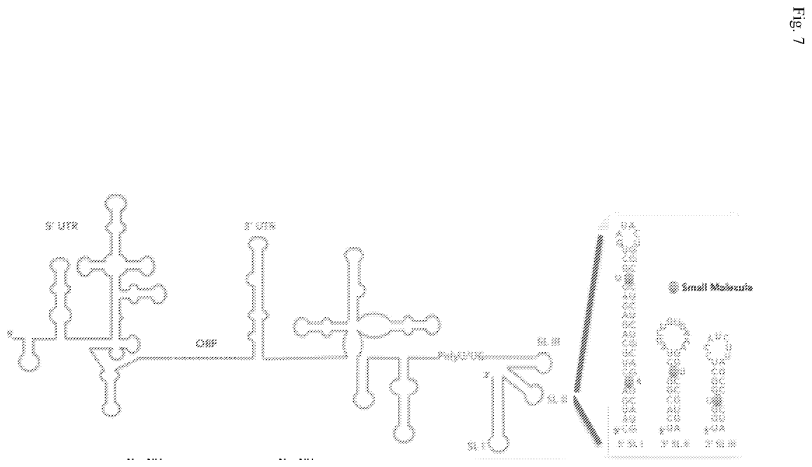

Of the 26,624 possible sequences in the five RNA libraries, 1,215 sequences (4.6%) are unique for a single compound; that is, the sequence only appears in the highest 0.5% of Z_{obs} values for one compound. The most selective RNA motifs were evaluated by searching for their sequences in the highest 0.5% and lowest 0.5% of Z_{obs} values. A selective RNA motif will be enriched for only a single or a few compounds and discriminated against by many compounds. The most selective motifs for libraries B-E are (FIG. 6): 5′CGAUUU3′ (discriminated against by nine compounds; privileged for one compound); 5′CCA3′/3′UC5′ (discriminated against by four compounds; privileged for one compound); 5′AAC3′/3′UAU′ (discriminated against by ten compounds; privileged for one compound) and 5′CCC3′/3′CCA5′ (discriminated against by nine compounds; privileged for one compound); and 5′UGGU3′/3′UGU5′ (discriminated against by nine compounds; privileged for one compound), respectively. Notably, there were no RNA motifs from library A that appeared in the highest 0.5% for few compounds and the lowest 0.5% for others.

The most promiscuous binding sequence for RNA library A is 5′GGUGU3′ (n=33 out of 38 total compounds) (FIG. 6). Other 5-nucleotide hairpins were discriminated against by many compounds, that is they do not bind, including 5′UUUUU3′ (n=35; does not appear in the highest 0.5% Z_{obs} values for any compound), 5′UCUUU3′ (n=35; does not appear in the highest 0.5% Z_{obs} values for any compound), 5′UUUUC3′ (n=36; does not appear in the highest 0.5% Z_{obs} values for any compound) (FIG. 6). Interestingly, all three sequences are U rich.

There are also 6-nucleotide hairpins (derived from RNA library B) that accommodate binding to many small molecules (FIG. 6): 5′CUAUAU3′ (n=55 out of 65 compounds), 5′UAAGAG3′ (n=56), 5′UACCUG3′ (n=61), 5′AAAUAA3′ (n=64), 5′UUAUAU3′ (n=64), 5′UAAUAU3′ (n=65), 5′UGCGUG3′ (n=65). Likewise, RNA motifs that do not generally form structures that bind the small molecules studied herein are (appear in the lowest 0.05% of Z_{obs} values): 5′ACCCAU3′ (n=54), 5′UCCAUU3′ (n=43); 5′CACACU3′ (n=44); 5′CUCAAU3′ (n=46); and 5′UCCUAU3′ (n=46) (FIG. 6).

Interestingly, the most promiscuous sequences from RNA library C are all predicted to fold into single nucleotide U bulges (FIG. 6): 5′GUU3′/3′C_G5′ (n=11 out of 32 compounds), 5′GGU(U)3′/3′CG_(A)5′ (n=8; where invariant nucleotides from the cassette are in parentheses), 5′GCU(U)3′-3′CG_(A)5′ (n=6; where invariant nucleotides from the cassette are in parentheses), 5′GUA3′/5′C_U3′ (n=6), 5′GUU3′/3′C_A5′ (n=6). RNAs 5′CGU3′/3′UU5′ (n=8 compounds), 5′CGU3′/3′GU5′ (n=7), 5′CGA3′/3′GU5′ (n=7) are the most highly discriminated against for binding (FIG. 6).

As observed for the other RNA libraries, there are internal loops derived from D that bind a wide range of small molecules (FIG. 6). They include 5′ACA3′/3′CGG5′ (n=46 out of 68 compounds), 5′GCU3′/3′CUG5′ (n=46), 5′ACA3′/3′CGC5′ (n=47), 5′ACU3′/3′CGU5′ (n=47), 5′CAA3′/3′CGG5′ (n=47), 5′GCU3′/3′CAC5′ (n=47), 5′GGU3′/3′CCC5′ (n=47), 5′UAU3′/3′CCC5′ (n=47). Many of these loops predicted to form loops with a U nucleotide opposite a C nucleotide. In contrast, 5′AAC3′/3′UAA5′ (n=27), 5′ACA3′/3′CAA5′ (n=30), 5′AAC3′/3′AAC5′ (n=31), and 5′CAU3′/3′CAA5′ (n=32) do not fold into structures that accommodate binding to the small molecules studied herein (FIG. 6).

The loops derived from E that bind the most number of compounds are 5′GCUU(U)3′/3′CGC(A)5′ (n=12 out of 30 compounds; where invariant nucleotides from the cassette are in parentheses), 5′GGUC3′/3′CCU5′ (n=12), and 5′GUGG(U)3′/3′CGC_(A)5′ (n=12) (FIG. 6). The two most highly discriminated against loops selected from library 5 are: 5′CGAU3′/3′GGG5′ (n=12) and 5′CGUG3/′3′UGG5′ (n=12) (FIG. 6). Collectively, it appears that pyrimidine nucleotides provide scaffolds for binding to small molecules in the context of 6-nucleotide hairpins (derived from B), bulges (derived from C), and internal loops (derived from C-E), perhaps due to their smaller size as compared to purines. This trend does not hold for 5-nucleotide hairpins (derived from A), which could fold into structures that are too small to accommodate binding.

Affinity of Selected RNA Motif-Small Molecule Interactions. The affinity of exemplar compounds that have inherent fluorescence was measured. As shown in Table 1, compounds 7 and 8 (FIG. 7A) bind to selected RNAs with affinities ranging from ˜400 nM-˜700 nM, >10-fold more tightly than non-selected RNAs and a fully base paired RNA control. Motifs with Z_{obs}<8 bind with much weaker affinity or do not bind. Indeed, the RNA motifs selected for compounds 7 and 8 only bind with high affinity if Z_{obs}>8 (Table 1), in agreement with previous studies¹², indicating that this cut-off is likely general. Binding affinities were confirmed using a label-free method, biolayer interferometry. In agreement with the fluorescence-based experiments, 7 and 8 bind RNAs with Z_{obs}>8 at least 10-fold more tightly than motifs with Z_{obs}<8 and base paired control RNAs.

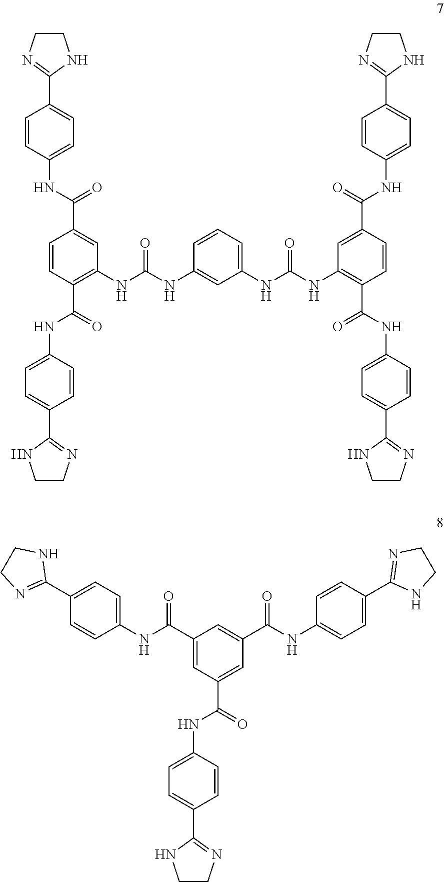 [SMILES: O=C(Nc1cccc(NC(=O)Nc2cc(C(=O)Nc3ccc(C4=NCCN4)cc3)ccc2C(=O)Nc2ccc(C3=NCCN3)cc2)c1)Nc1cc(C(=O)Nc2ccc(C3=NCCN3)cc2)ccc1C(=O)Nc1ccc(C2=NCCN2)cc1 ; O=C(Nc1ccc(C2=NCCN2)cc1)c1cc(C(=O)Nc2ccc(C3=NCCN3)cc2)cc(C(=O)Nc2ccc(C3=NCCN3)cc2)c1]

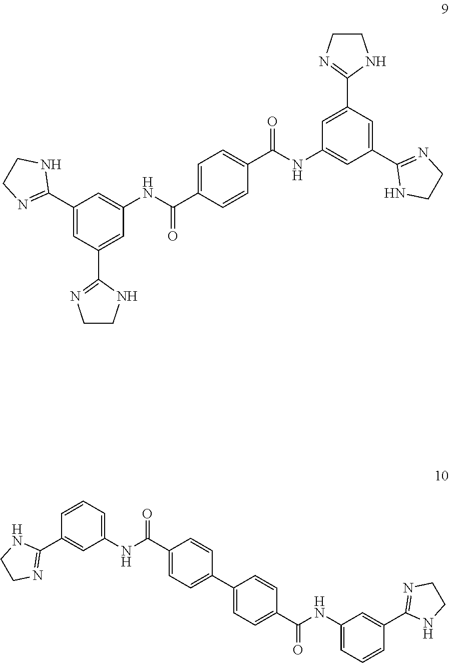 [SMILES: O=C(Nc1cc(C2=NCCN2)cc(C2=NCCN2)c1)c1ccc(C(=O)Nc2cc(C3=NCCN3)cc(C3=NCCN3)c2)cc1 ; O=C(Nc1cccc(C2=NCCN2)c1)c1ccc(-c2ccc(C(=O)Nc3cccc(C4=NCCN4)c3)cc2)cc1]

Scaffold and Chemoinformatics Analyses of Hit Compounds. To aid in the design of small molecules that bind RNA and to assess rigorously their drug-likeness, scaffold and chemoinformatics analyses of hit compounds were carried out. The 91 compounds that bind the RNA libraries vary in similarity to the starting lead small molecules: H1, 0.30±0.13; range: 0.09-0.75; 1a, 0.26±0.06; range: 0.12-0.48; and D6, 0.21±0.04; range: 0.11-0.30. Comparison of the hit compounds to each other affords Tanimoto scores ranging from 0.09 to 0.97, suggesting that chemical diversity and common scaffolds are found amongst the small molecules.

To gain general insight into the chemotypes that confer avidity for RNA, sub-features in the 91 hit compounds were identified via the method of Clark and Labute¹³. (Table 2). To quantify the significance of the sub-structures, a pooled population comparison was carried out on how frequently the sub-structure appears in the hit compounds and how frequently the same sub-structure appears in the 1,987 compounds in the small molecule library. This statistical analysis calculates Z_{obs}, and hence p-values, for each sub-structure, akin to the analysis of statistically significant privileged motifs. This analysis afforded 11 privileged chemotypes, with S3 being the most statistically significant sub-structure for conferring avidity for RNA (Table 2).

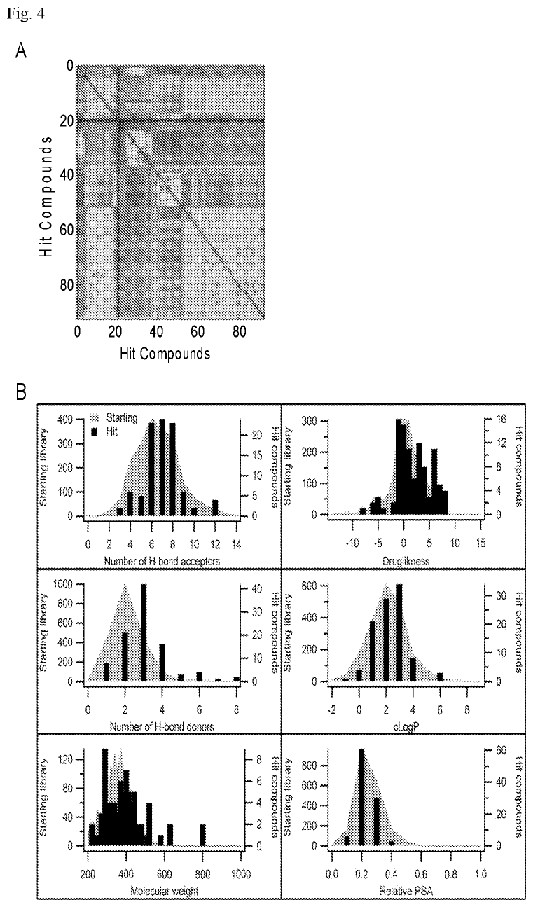

Interestingly, the chemotypes identified here are present in previously discovered RNA small molecule binders (Table 2). For example, S6 is found in two molecules used to target trinucleotide repeat expansions that cause myotonic dystrophy type 1 and type 2^{38, 39}. S3 is found in small molecules that were developed to target the trans-activation response element (TAR RNA) of human immunodeficiency virus 1 (HIV-1)⁴⁰ while S13 is found in a compound that binds the aminoacyl-tRNA acceptor site (A-site) of Escherichia coli ribosome⁴¹. S5 was previously identified as a privileged chemotype for binding RNA¹¹. Drug-Like Properties Analysis of Hit Compounds. Compared to FDA-approved drugs, the hit compounds have similar distribution of Log P values, Log D values, molecular weights and number of rotatable bonds (FIG. 4). Interestingly, the hit compounds have Log D values between −4.4 and 7.1, with the majority having Log D values between 1-3 (average=1.85±1.55; median=2.22). The starting compound library has Log D between −4.4-9.2 with the majority having Log D values between 3-4 (average=2.70±1.79; median=2.94). Therefore, the hit compounds are more similar in lipophilicity to compounds in DrugBank than the starting library is, suggesting that Log D values can be used as a molecular parameter that promotes the binding of small molecules to RNA.

Lipinski parameters⁴² were computed for previously published small molecules that were studied by 2DCS including eight benzimidazoles and five 2-aminobenzimidazoles^{11, 35, 36}. This small set of compounds also has similar c Log P values (range: 2-3), c Log D values (range: 0-3) range and number of H-bond donors (3) as that of the hit compounds (FIG. 4). However, the majority of the published small molecules have higher molecular weights (500-600 g/mol vs. 300-400), number of H-bond acceptors (8 vs. 6) and number of rotatable bonds (16 vs. 4-7) than the hit compounds and FDA-approved drugs (FIG. 4). Taken together, the hit compounds bind to members of the RNA libraries specifically and are drug-like.

Biological activity of exemplar compounds. Inforna has informed the design of compounds that target various disease-associated RNAs including, expanded repeating RNAs that cause microsatellite disorders and oncogenic microRNAs. Therefore, the Inforna approach was expanded to other types of RNAs, in particular viral RNAs. Interestingly, related compounds of the phenylimidazoline class were predicted to bind bulges present in the hepatitis C Virus (HCV) 3′ untranslated region (UTR) (FIG. 7A). HCV is a member of the Flaviridae family of viruses (also includes West Nile and Zika viruses) which are comprised of a single open reading frame (ORF) that encodes all viral proteins, flanked by structured 5′ and 3′ UTRs (FIG. 7A). Viral replication initiates at the 3′ UTR, which contains conserved RNA structures that appear to be required for replication to occur⁴³⁻⁴⁸. These include a 3′ stem-loop (3′ SL)⁴⁸⁻⁵² and several upstream stem-loops (SL I-SL III; FIG. 7A)) that contain cyclization sequences (CS)⁵⁰ that are complementary to the 5′ end of viral RNA′. Not surprisingly, the complementary nucleotides at the 5′ end of the viral RNA are also conserved (SLA), which participate in long range-interactions with the 3′ UTR⁵⁴⁻⁵⁶.

The importance of long range RNA-RNA interactions required for the replication of HCV suggests that stable RNA structures of defined composition exist, and the disclosed methods have identified molecules that bind to some of these structures. The compounds bind their cognate loops in the 3′ UTR with mid-nanomolar to low micromolar affinities (Table 1). It was next determined if the identified compounds could inhibit HCV RNA replication in HCV subgenomic replicon cells, in particular SGR-Neo-RLuc-JFH1-2A replicon cells established in Huh-7.5 (human hepatoma cells). In this system, an autonomously replicating viral RNA replicon is used to monitor efficiently RNA replication of the virus in the absence of spread of viral RNA to adjacent cells. This is achieved by replacing the structural proteins required to assembly progeny virions with a drug selectable marker (Neo) and a Renilla luciferase (RLuc) reporter gene. Once this replicon has been delivered to human hepatoma cells and selection with G418 (conferred by Neo selectable marker) has occurred, the expression and Renilla luciferase activity is directly correlated with the level of HCV replication⁵⁷.

Inforna identified three lead compounds for HCV RNA that bind motifs in SL1 and SLII, compounds 7, 8, and 10 with varying affinities (Table 1; FIG. 7B). A mutational study showed that thermodynamically stable structural elements in SL I and SL II are required for viral replication⁵⁸, suggesting that small molecule targeting of these elements could be inhibitory. They were therefore assayed for their ability to inhibit HCV replication using this replicon system, with DMSO vehicle and 2′-C-methyladenosine¹⁷ triphosphate serving as negative and positive controls, respectively. A structurally similar 9 was also tested, which does not bind SLI and SLII motifs and thus serves as a negative control (Table 1). Indeed, dose-dependent inhibition was observed for 7, 8, and 10 with IC₅₀'s of 17±0.5, 14±1, and 19±2 μM, respectively (FIG. 7C). None of the compounds were toxic to Huh-7.5 cells, as determined by a cell viability assay. Notably, 9 does not inhibit HCV replication with an IC₅₀>100 □M. The differences between in vitro binding affinity and cellular potency could be due to a variety of factors including cellular uptake and localization, amongst others.

Interestingly, 7, 8, and 10 have potencies similar to 2′-C-methyladenosine (IC₅₀=4.5±0.1 μM), which inhibits HCV transcription via competitive inhibition of the HCV RNA polymerase nonstructural protein 5B (NS5B)¹⁷. Here, an inhibitor was designed with similar potency that binds the viral RNA rather than a viral enzyme.

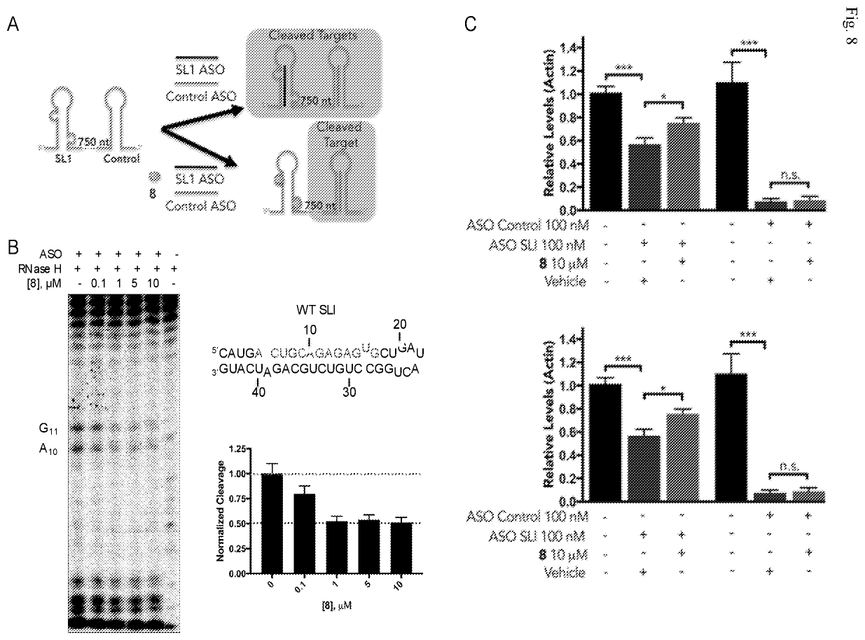

Investigating Compound Mode of Action. Many of the compounds identified from the disclosed screen that bind RNA selectively share chemotypes with kinase inhibitors. To further support RNA binding as a mode of action for inhibition of HCV replication, an antisense oligonucleotide-based approach was developed to study compound binding sites in cells (FIG. 8); an in-depth study of the abilities of 7-10 to inhibit kinases was completed; and dozens of kinase inhibitors were studied to see if they affect HCV. In summary, each data set supports RNA binding as a mode of action for the compounds.

Compound 8 binds the desired site in HCV RNA in vitro and in cells. To validate that the lead compounds bind to the desired site in the HCV RNA genome, an approach to profile binding site by using an antisense oligonucleotide was developed (FIG. 8A). Antisense oligonucleotides bind to complementary RNAs and recruit RNase H, which cleaves the RNA strand. Upon small molecule binding, the RNA's structure is stabilized, making it more difficult for the oligonucleotide to invade the structure. Thus, small molecule binding sites can be read out by inhibition of oligonucleotide cleavage. For in vitro studies, a small segment of the target site was transcribed that folds into the desired structure.

Application of an antisense agent (Table 4) and RNase H cleaves the RNA at the desired site. Addition of compound 8 to these reactions limited the ability of the antisense to mediate cleavage of the target (FIG. 8B). Additional control experiments were completed in which the RNA was mutated to remove 8's binding site and the corresponding complementary oligonucleotide to induce RNase H cleavage. As expected, the extent and pattern of cleavage were similar in the absence and the presence of compound 8.

Given these favorable results in vitro, the competitive cleaving approach was used to map cellular ligand binding sites. Indeed, compound 8 inhibited cleavage induced by an antisense oligonucleotide that overlaps with the 8-binding site, as determined by RT-qPCR and supported by readout of viral replication using luciferase (synergistic effect of compound and ASO treatment) (FIG. 8C). Importantly, 8 does not rescue cleavage using an oligonucleotide that is complementary to a downstream site that does not have an 8-binding site, as determined by RT-qPCR (“Control ASO”; FIG. 8C, top). Previous approaches to map small molecule-RNA binding sites, such as Chemical Cross-Linking and Isolation by Pull-down (Chem-CLIP)59-61 and Small Molecule Nucleic Acid Profiling by Cleavage Applied to RNA (Ribo-SNAP)61, require the synthesis of derivatives of the lead molecules that allow them to cross-link or cleave and RNA target, respectively. The oligonucleotide profiling approach described herein does not require additional chemical synthesis, but rather uses oligonucleotide cleavage and competing this cleavage at specific sites with a small molecule.
1. Guan, L. and Disney, M. D. (2012). Recent advances in developing small molecules targeting RNA. ACS Chem. Biol. 7, 73-86.
2. Thomas, J. R. and Hergenrother, P. J. (2008). Targeting RNA with small molecules. Chem. Rev. 108, 1171-1224.
3. Blount, K. F. and Breaker, R. R. (2006). Riboswitches as antibacterial drug targets. Nat. Biotechnol. 24, 1558-1564.
4. Tenson, T. and Mankin, A. (2006). Antibiotics and the ribosome. Mol. Microbiol. 59, 1664-1677.
5. Schlunzen, F., Zarivach, R., Harms, J., Bashan, A., Tocilj, A., Albrecht, R., Yonath, A. and Franceschi, F. (2001). Structural basis for the interaction of antibiotics with the peptidyl transferase centre in eubacteria. Nature 413, 814-821.
6. Hermann, T. (2016). Small molecules targeting viral rna. Wiley Interdiscip. Rev. RNA 7, 726-743.
7. Ohgushi, M., Kuroki, S., Fukamachi, H., O'Reilly, L. A., Kuida, K., Strasser, A. and Yonehara, S. (2005). Transforming growth factor beta-dependent sequential activation of smad, bim, and caspase-9 mediates physiological apoptosis in gastric epithelial cells. Mol. Cell. Biol. 25, 10017-10028.
8. Velagapudi, S. P., Gallo, S. M. and Disney, M. D. (2014). Sequence-based design of bioactive small molecules that target precursor micrornas. Nat. Chem. Biol. 10, 291-297.
9. Disney, M. D., Winkelsas, A. M., Velagapudi, S. P., Southern, M., Fallahi, M. and Childs-Disney, J. L. (2016). Inforna 2.0: A platform for the sequence-based design of small molecules targeting structured rnas. ACS Chem. Biol. 11, 1720-1728.
10. Disney, M. D., Labuda, L. P., Paul, D. J., Poplawski, S. G., Pushechnikov, A., Tran, T., Velagapudi, S. P., Wu, M. and Childs-Disney, J. L. (2008). Two-dimensional combinatorial screening identifies specific aminoglycoside-rna internal loop partners. J. Am. Chem. Soc. 130, 11185-11194.
11. Tran, T. and Disney, M. D. (2012). Identifying the preferred rna motifs and chemotypes that interact by probing millions of combinations. Nat. Commun. 3, 1125.
12. Velagapudi, S. P., Luo, Y., Tran, T., Haniff, H. S., Nakai, Y., Fallahi, M., Martinez, G. J., Childs-Disney, J. L. and Disney, M. D. (2017). Defining rna-small molecule affinity landscapes enables design of a small molecule inhibitor of an oncogenic noncoding RNA. ACS Cent. Sci. 3, 205-216.
13. Clark, A. M. and Labute, P. (2009). Detection and assignment of common scaffolds in project databases of lead molecules. J. Med. Chem. 52, 469-483.
14. Bevilacqua, J. M. and Bevilacqua, P. C. (1998). Thermodynamic analysis of an RNA combinatorial library contained in a short hairpin. Biochemistry 37, 15877-15884.
15. Paul, D. J., Seedhouse, S. J. and Disney, M. D. (2009). Two-dimensional combinatorial screening and the RNA privileged space predictor program efficiently identify aminoglycoside-RNA hairpin loop interactions. Nucleic Acids Res. 37, 5894-5907.
16. Nettling, M., Treutler, H., Grau, J., Keilwagen, J., Posch, S. and Grosse, I. (2015). Difflogo: A comparative visualization of sequence motifs. BMC Bioinformatics 16, 387.
17. Carroll, S. S., Tomassini, J. E., Bosserman, M., Getty, K., Stahlhut, M. W., Eldrup, A. B., Bhat, B., Hall, D., Simcoe, A. L., LaFemina, R., Rutkowski, C. A., Wolanski, B., Yang, Z., Migliaccio, G., De Francesco, R., Kuo, L. C., MacCoss, M. and Olsen, D. B. (2003). Inhibition of hepatitis c virus rna replication by 2′-modified nucleoside analogs. J. Biol. Chem. 278, 11979-11984.
18. Disney, M. D., Liu, B., Yang, W.-Y., Sellier, C., Tran, T., Charlet-Berguerand, N. and Childs-Disney, J. L. (2012). A small molecule that targets r(CGG)^{exp} and improves defects in fragile x-associated tremor ataxia syndrome. ACS Chem. Biol. 7, 1711-1718.
19. Pushechnikov, A., Lee, M. M., Childs-Disney, J. L., Sobczak, K., French, J. M., Thornton, C. A. and Disney, M. D. (2009). Rational design of ligands targeting triplet repeating transcripts that cause RNA dominant disease: Application to myotonic muscular dystrophy type 1 and spinocerebellar ataxia type 3. J. Am. Chem. Soc. 131, 9767-9779.
20. Parkesh, R., Childs-Disney, J. L., Nakamori, M., Kumar, A., Wang, E., Wang, T., Hoskins, J., Housman, D. E., Thornton, C. A., Disney, M. D. and Tran, T. (2012). Design of a bioactive small molecule that targets the myotonic dystrophy type 1 rna via an RNA motif-ligand database & chemical similarity searching. J. Am. Chem. Soc. 134, 4731-4742.
21. Kumar, A., Parkesh, R., Sznajder, L. J., Childs-Disney, J. L., Sobczak, K. and Disney, M. D. (2012). Chemical correction of pre-mRNA splicing defects associated with sequestration of muscleblind-like 1 protein by expanded r(CAG)-containing transcripts. ACS Chem. Biol. 3, 496-505.
22. Frazier, K. S. (2015). Antisense oligonucleotide therapies: The promise and the challenges from a toxicologic pathologist's perspective. Toxicol. Pathol. 43, 78-89.
23. Willett, P. (2011). Similarity searching using 2D structural fingerprints. Methods Mol. Biol. 672, 133-158.
24. Wishart, D. S., Knox, C., Guo, A. C., Shrivastava, S., Hassanali, M., Stothard, P., Chang, Z. and Woolsey, J. (2006). Drugbank: A comprehensive resource for in silico drug discovery and exploration. Nucleic Acids Res. 34, D668-672.
25. Wishart, D. S., Knox, C., Guo, A. C., Cheng, D., Shrivastava, S., Tzur, D., Gautam, B. and Hassanali, M. (2008). Drugbank: A knowledgebase for drugs, drug actions and drug targets. Nucleic Acids Res. 36, D901-906.
26. Knox, C., Law, V., Jewison, T., Liu, P., Ly, S., Frolkis, A., Pon, A., Banco, K., Mak, C., Neveu, V., Djoumbou, Y., Eisner, R., Guo, A. C. and Wishart, D. S. (2011). Drugbank 3.0: A comprehensive resource for ‘omics’ research on drugs. Nucleic Acids Res. 39, D1035-1041.
27. Hou, T. J. and Xu, X. J. (2003). ADME evaluation in drug discovery. 2. Prediction of partition coefficient by atom-additive approach based on atom-weighted solvent accessible surface areas. J. Chem. Inf. Comput. Sci. 43, 1058-1067.
28. Viswanadhan, V. N., Ghose, A. K., Revankar, G. R. and Robins, R. K. (1989). Atomic physicochemical parameters for 3 dimensional structure directed quantitative structure—activity relationships .4. Additional parameters for hydrophobic and dispersive interactions and their application for an automated superposition of certain naturally-occurring nucleoside antibiotics. J. Chem. Inf. Comput. Sci. 29, 163-172.
29. Klopman, G., Li, J. Y., Wang, S. M. and Dimayuga, M. (1994). Computer automated log p calculations based on an extended group-contribution approach. J J. Chem. Inf. Comput. Sci. 34, 752-781.
30. Csizmadia, F., TsantiliKakoulidou, A., Panderi, I. and Darvas, F. (1997). Prediction of distribution coefficient from structure .1. Estimation method. J. Pharm. Sci. 86, 865-871.
31. Rutkowska, E., Pajak, K. and Jozwiak, K. (2013). Lipophilicity—methods of determination and its role in medicinal chemistry. Acta Pol. Pharm 0.70, 3-18.
32. Leo, A., Hansch, C. and Elkins, D. (1971). Partition coefficients and their uses. Chem. Rev. 71, 525-+.
33. Barbato, F., Caliendo, G., Larotonda, M. I., Silipo, C., Toraldo, G. and Vittoria, A. (1986). Distribution coefficients by curve fitting—application to ionogenic nonsteroidal antiinflammatory drugs. Quant. Struct.-Act. Rel. 5, 88-95.
34. Disney, M. D. and Childs-Disney, J. L. (2007). Using selection to identify and chemical microarray to study the RNA internal loops recognized by 6′-n-acylated kanamycin A. Chembiochem 8, 649-656.
35. Velagapudi, S. P., Pushechnikov, A., Labuda, L. P., French, J. M. and Disney, M. D. (2012). Probing a 2-aminobenzimidazole library for binding to RNA internal loops via two-dimensional combinatorial screening. ACS Chem. Biol. 7, 1902-1909.
36. Velagapudi, S. P., Seedhouse, S. J., French, J. and Disney, M. D. (2011). Defining the RNA internal loops preferred by benzimidazole derivatives via 2D combinatorial screening and computational analysis. J. Am. Chem. Soc. 133, 10111-10118.
37. Schneider, T. D. and Stephens, R. M. (1990). Sequence logos: A new way to display consensus sequences. Nucleic Acids Res. 18, 6097-6100.
38. Gareiss, P. C., Sobczak, K., McNaughton, B. R., Palde, P. B., Thornton, C. A. and Miller, B. L. (2008). Dynamic combinatorial selection of molecules capable of inhibiting the (CUG) repeat RNA-MBNL1 interaction in vitro: Discovery of lead compounds targeting myotonic dystrophy (DM1). J. Am. Chem. Soc. 130, 16254-16261.
39. Wong, C.-H., Fu, Y., Ramisetty, S. R., Baranger, A. M. and Zimmerman, S. C. (2011). Selective inhibition of mbn1l-ccug interaction by small molecules toward potential therapeutic agents for myotonic dystrophy type 2 (DM2). Nucleic Acids Res. 39, 8881-8890.
40. Murchie, A. I., Davis, B., Isel, C., Afshar, M., Drysdale, M. J., Bower, J., Potter, A. J., Starkey, I. D., Swarbrick, T. M., Mirza, S., Prescott, C. D., Vaglio, P., Aboul-ela, F. and Karn, J. (2004). Structure-based drug design targeting an inactive rna conformation: Exploiting the flexibility of HIV-1 TAR RNA. J. Mol. Biol. 336, 625-638.
41. Foloppe, N., Chen, I. J., Davis, B., Hold, A., Morley, D. and Howes, R. (2004). A structure-based strategy to identify new molecular scaffolds targeting the bacterial ribosomal A-site. Bioorg. Med. Chem. 12, 935-947.
42. Lipinski, C. A., Lombardo, F., Dominy, B. W. and Feeney, P. J. (2001). Experimental and computational approaches to estimate solubility and permeability in drug discovery and development settings. Adv. Drug Deliv. Rev. 46, 3-26.
43. Elghonemy, S., Davis, W. G. and Brinton, M. A. (2005). The majority of the nucleotides in the top loop of the genomic 3′ terminal stem loop structure are cis-acting in a West Nile virus infectious clone. Virology 331, 238-246.
44. Khromykh, A. A., Kondratieva, N., Sgro, J. Y., Palmenberg, A. and Westaway, E. G. (2003). Significance in replication of the terminal nucleotides of the flavivirus genome. J. Virol. 77, 10623-10629.
45. Tilgner, M., Deas, T. S. and Shi, P. Y. (2005). The flavivirus-conserved penta-nucleotide in the 3′ stem-loop of the West Nile virus genome requires a specific sequence and structure for rna synthesis, but not for viral translation. Virology 331, 375-386.
46. Tilgner, M. and Shi, P. Y. (2004). Structure and function of the 3′ terminal six nucleotides of the West Nile virus genome in viral replication. J. Virol. 78, 8159-8171.
47. Yu, L. and Markoff, L. (2005). The topology of bulges in the long stem of the flavivirus 3′ stem-loop is a major determinant of rna replication competence. J. Virol. 79, 2309-2324.
48. Zeng, L., Falgout, B. and Markoff, L. (1998). Identification of specific nucleotide sequences within the conserved 3′-SL in the dengue type 2 virus genome required for replication. J. Virol. 72, 7510-7522.
49. Brinton, M. A., Fernandez, A. V. and Dispoto, J. H. (1986). The 3′-nucleotides of flavivirus genomic rna form a conserved secondary structure. Virology 153, 113-121.
50. Men, R., Bray, M., Clark, D., Chanock, R. M. and Lai, C. J. (1996). Dengue type 4 virus mutants containing deletions in the 3′ noncoding region of the rna genome: Analysis of growth restriction in cell culture and altered viremia pattern and immunogenicity in rhesus monkeys. J. Virol. 70, 3930-3937.
51. Proutski, V., Gould, E. A. and Holmes, E. C. (1997). Secondary structure of the 3′ untranslated region of flaviviruses: Similarities and differences. Nucleic Acids Res. 25, 1194-1202.
52. Rauscher, S., Flamm, C., Mandl, C. W., Heinz, F. X. and Stadler, P. F. (1997). Secondary structure of the 3′-noncoding region of flavivirus genomes: Comparative analysis of base pairing probabilities. RNA 3, 779-791.
53. Hahn, C. S., Hahn, Y. S., Rice, C. M., Lee, E., Dalgarno, L., Strauss, E. G. and Strauss, J. H. (1987). Conserved elements in the 3′ untranslated region of flavivirus rnas and potential cyclization sequences. J Mol. Biol. 198, 33-41.
54. Khromykh, A. A., Meka, H., Guyatt, K. J. and Westaway, E. G. (2001). Essential role of cyclization sequences in flavivirus rna replication. J. Virol. 75, 6719-6728.
55. Lo, M. K., Tilgner, M., Bernard, K. A. and Shi, P. Y. (2003). Functional analysis of mosquito-borne flavivirus conserved sequence elements within 3′ untranslated region of west nile virus by use of a reporting replicon that differentiates between viral translation and rna replication. J. Virol. 77, 10004-10014.
56. Alvarez, D. E., Lodeiro, M. F., Luduena, S. J., Pietrasanta, L. I. and Gamarnik, A. V. (2005). Long-range RNA-RNA interactions circularize the dengue virus genome. J. Virol. 79, 6631-6643.
57. Targett-Adams, P. and McLauchlan, J. (2005). Development and characterization of a transient-replication assay for the genotype 2a hepatitis C virus subgenomic replicon. J. Gen. Virol. 86, 3075-3080.
58. Friebe, P. and Bartenschlager, R. (2009). Role of RNA structures in genome terminal sequences of the hepatitis C virus for replication and assembly. J. Virol. 83, 11989-11995.
59. Yang, W.-Y., Wilson, H. D., Velagapudi, S. P. and Disney, M. D. (2015). Inhibition of non-atg translational events in cells via covalent small molecules targeting RNA. J. Am. Chem. Soc. 137, 5336-5345.
60. Guan, L. and Disney, M. D. (2013). Covalent small-molecule-RNA complex formation enables cellular profiling of small-molecule-RNA interactions. Angew. Chem. Int. Ed. Engl. 52, 10010-10013.
61. Rzuczek, S. G., Colgan, L. A., Nakai, Y., Cameron, M. D., Furling, D., Yasuda, R. and Disney, M. D. (2016). Precise small-molecule recognition of a toxic CUG rna repeat expansion. Nat. Chem. Biol. 13, 188. 62. Colak, D., Zaninovic, N., Cohen, M. S., Rosenwaks, Z., Yang, W. Y., Gerhardt, J., Disney, M. D. and Jaffrey, S. R. (2014). Promoter-bound trinucleotide repeat mrna drives epigenetic silencing in fragile X syndrome. Science 343, 1002-1005.
63. Rzuczek, S. G., Park, H. and Disney, M. D. (2014). A toxic RNA catalyzes the in cellulo synthesis of its own inhibitor Angew Chem Int Ed Engl 53, 10956-10959.
64. Su, Z., Zhang, Y., Gendron, T. F., Bauer, P. O., Chew, J., Yang, W. Y., Fostvedt, E., Jansen-West, K., Belzil, V. V., Desaro, P., Johnston, A., Overstreet, K., Oh, S. Y., Todd, P. K., Berry, J. D., Cudkowicz, M. E., Boeve, B. F., Dickson, D., Floeter, M. K., Traynor, B. J., Morelli, C., Ratti, A., Silani, V., Rademakers, R., Brown, R. H., Rothstein, J. D., Boylan, K. B., Petrucelli, L. and Disney, M. D. (2014). Discovery of a biomarker and lead small molecules to target r(GGGGCC)-associated defects in c9FTD/ALS. Neuron 83, 1043-1050.
65. Velagapudi, S. P., Cameron, M. D., Haga, C. L., Rosenberg, L. H., Lafitte, M., Duckett, D. R., Phinney, D. G. and Disney, M. D. (2016). Design of a small molecule against an oncogenic noncoding RNA. Proc. Natl. Acad. Sci. U.S.A. 113, 5898-5903.

All patents and publications referred to herein are incorporated by reference herein to the same extent as if each individual publication was specifically and individually indicated to be incorporated by reference in its entirety.

TABLE 1
Affinities of selected RNA motif-small molecule interactions derived from selections
with RNA library C and small molecule affinities for HCV RNA Motifs.

| RNA motif-small molecule interactions derived from selections with RNA library C | | | | | | HCV RNA Motifs | | |
|---|---|---|---|---|---|---|---|---|
| | Small | Z_{obs} | | Fitness | Affinity | | Small | Affinity |
| RNA Motif | Molecule | (Rank) | | Score | (nM) | RNA Motif | Molecule | (nM) |
| 5′UUA3′/3′A_U5′ | 7 | 25.2 | (1) | 100 | 380 ± 26 | 5′GUG3′/3′C_C5′ | 7 | 470 ± 32 |
| | | | | | | (SL I and SL III) | | |
| 5′GAG3′/3′C_C5′ | 7 | 18.3 | (20) | 73 | 410 ± 26 | | 8 | 1400 ± 160 |
| 5′UGU3′/3′G_A5′ | 7 | 11.8 | (49) | 47 | 630 ± 36 | | 9 | N.B. |
| 5′AAC3′/3′U_G5′ | 7 | 8.2 | (94) | 33 | 420 ± 41 | | 10 | 3350 ± 770 |
| 5′UCCUU3′/ | 7 | 1.1 | (364) | N/A | 2080 ± 300 | 5′CUG3′/3′G_C5′ | 7 | 820 ± 82 |
| 3′AUUA5′ | | | | | | (SL II) | | |
| (non-binder) | | | | | | | | |
| 5′UCCAU3′/3′AUCA | 7 | −2.8 | (436) | N/A | 7200 ± 418 | | 8 | 1400 ± 211 |
| (non-binder) | | | | | | | | |
| Fully paired RNA | 7 | N/A | | N/A | >7000 | | 9 | N.B. |
| | | | | | | | 10 | 7100 ± 4200 |
| 5′UUA3′/3′A_U5′ | 8 | 13.6 | (1) | 100 | 690 ± 47 | 5′UAG3′/3′A_C5′ | 7 | 460 ± 70 |
| | | | | | | (SL I) | | |
| 5′GAU3′/3′C_A5′ | 8 | 11.8 | (7) | 87 | 600 ± 61 | | 8 | 849 ± 150 |
| 5′GCU3′/3′C_A5′ | 8 | 10.8 | (14) | 79 | 450 ± 47 | | 9 | 1300 ± 500 |
| 5′UUA3′/3′A_U5′ | 8 | 9.5 | (24) | 70 | 530 ± 45 | | 10 | 460 ± 140 |
| 5′UCUAU3′/ | 8 | 6.1 | (396) | 45 | 5580 ± 440 | | | |
| 3′ACCA5′ | | | | | | | | |

| | | | | |
|---|---|---|---|---|
| Fully paired RNA | 8 | N/A | N/A | >5000 |

N.B. = no binding observed

TABLE 2
Chemotypees that confer avidity for RNA.

| | Number of hit | | | |
|---|---|---|---|---|
| | compounds / | | | % cpds in |
| | starting | | | DrugBank |
| | library with | | | with |
| Scaffold | chemotype | Z_{obs} | p-value | chemotype |
| 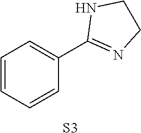 | 16 / 28 | 10.22 | <0.0001 | 0.0 |
| 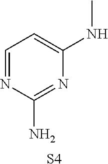 | 46 / 397 | 6.23 | <0.0001 | 0.4 |
| 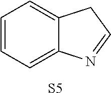 | 15 / 64 | 6.03 | <0.0001 | 4.4 |
| 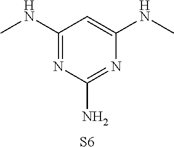 | 21 / 141 | 5.09 | <0.0001 | 0.04 |
| 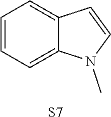 | 7 / 25 | 4.57 | <0.0001 | 1.5 |
| 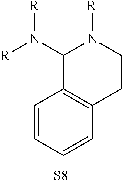 | 2 / 3 | 3.94 | 0.00008 | 0.0 |
| 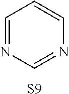 | 58 / 870 | 2.81 | 0.0048 | 13.9 |
| 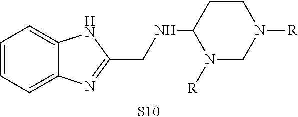 | 5 / 39 | 2.38 | 0.01928 | 0.0 |
| 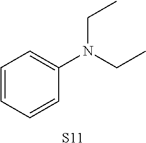 | 10 / 95 | 2.34 | 0.01928 | 2.4 |
| 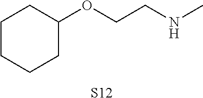 | 5 / 37 | 2.19 | 0.0285 | 0.1 |
| 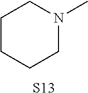 | 13 / 147 | 2.07 | 0.03845 | 4.0 |

TABLE 3

| | |
|---|---|
| 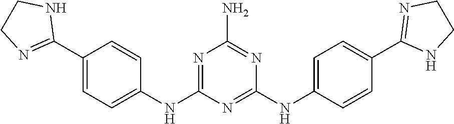 [SMILES: Nc1nc(Nc2ccc(C3=NCCN3)cc2)nc(Nc2ccc(C3=NCCN3)cc2)n1] | 1 (NCI-B10) |
| 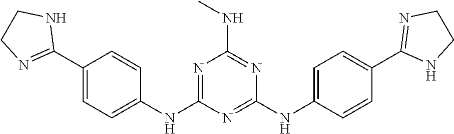 [SMILES: CNc1nc(Nc2ccc(C3=NCCN3)cc2)nc(Nc2ccc(C3=NCCN3)cc2)n1] | 2 (NCI-B11) |
| 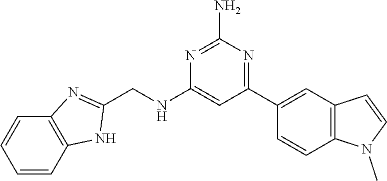 [SMILES: Cn1ccc2cc(-c3cc(NCc4nc5ccccc5[nH]4)nc(N)n3)ccc21] | 3 (LD-1038) |
| 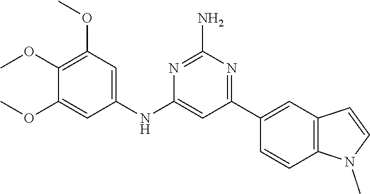 [SMILES: COc1cc(Nc2cc(-c3ccc4c(ccn4C)c3)nc(N)n2)cc(OC)c1OC] | 4 (LD-1028) |
| 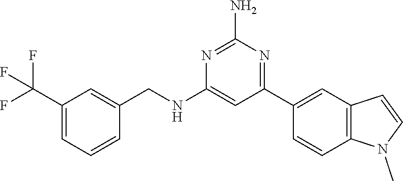 [SMILES: Cn1ccc2cc(-c3cc(NCc4cccc(C(F)(F)F)c4)nc(N)n3)ccc21] | 5 (LD-1036) |
| 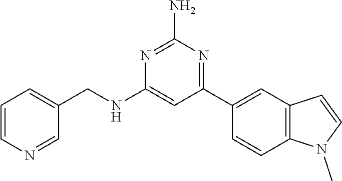 [SMILES: Cn1ccc2cc(-c3cc(NCc4cccnc4)nc(N)n3)ccc21] | 6 (LD-1064) |
| 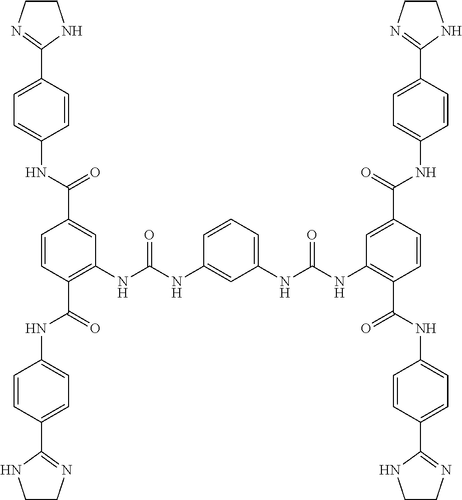 [SMILES: O=C(Nc1cccc(NC(=O)Nc2cc(C(=O)Nc3ccc(C4=NCCN4)cc3)ccc2C(=O)Nc2ccc(C3=NCCN3)cc2)c1)Nc1cc(C(=O)Nc2ccc(C3=NCCN3)cc2)ccc1C(=O)Nc1ccc(C2=NCCN2)cc1] | 7 (NCI-B17) |
| 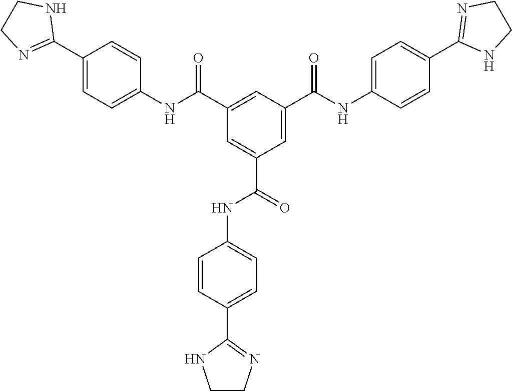 [SMILES: O=C(Nc1ccc(C2=NCCN2)cc1)c1cc(C(=O)Nc2ccc(C3=NCCN3)cc2)cc(C(=O)Nc2ccc(C3=NCCN3)cc2)c1] | 8 (NCI-B12) |
| 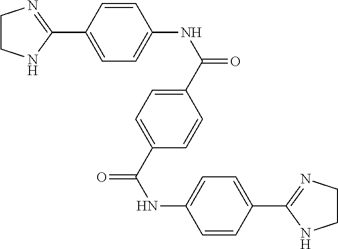 [SMILES: O=C(Nc1ccc(C2=NCCN2)cc1)c1ccc(C(=O)Nc2ccc(C3=NCCN3)cc2)cc1] | NCI-35843 |
| 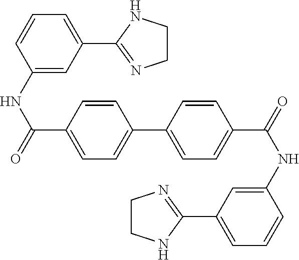 [SMILES: O=C(Nc1cccc(C2=NCCN2)c1)c1ccc(-c2ccc(C(=O)Nc3cccc(C4=NCCN4)c3)cc2)cc1] | NCI-50460 |
| 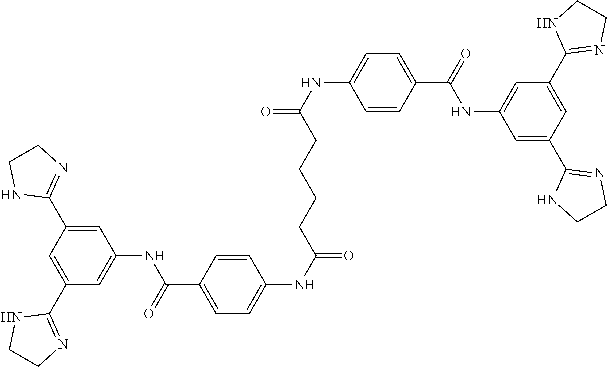 [SMILES: O=C(CCCCC(=O)Nc1ccc(C(=O)Nc2cc(C3=NCCN3)cc(C3=NCCN3)c2)cc1)Nc1ccc(C(=O)Nc2cc(C3=NCCN3)cc(C3=NCCN3)c2)cc1] | NCI-50462 |
| 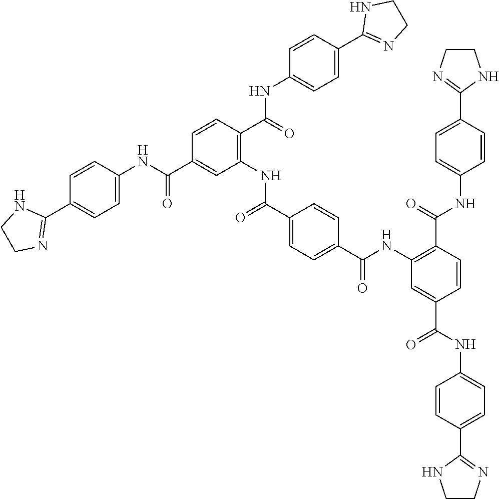 [SMILES: O=C(Nc1ccc(C2=NCCN2)cc1)c1ccc(C(=O)Nc2ccc(C3=NCCN3)cc2)c(NC(=O)c2ccc(C(=O)Nc3cc(C(=O)Nc4ccc(C5=NCCN5)cc4)ccc3C(=O)Nc3ccc(C4=NCCN4)cc3)cc2)c1] | NCI-A14 |
| 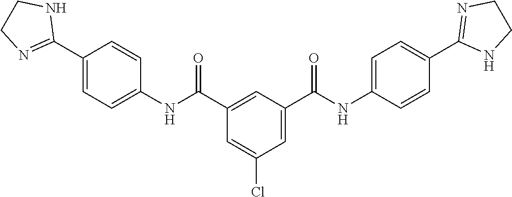 [SMILES: O=C(Nc1ccc(C2=NCCN2)cc1)c1cc(Cl)cc(C(=O)Nc2ccc(C3=NCCN3)cc2)c1] | NCI-A19 |
| 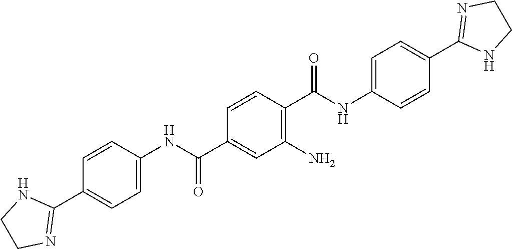 [SMILES: Nc1cc(C(=O)Nc2ccc(C3=NCCN3)cc2)ccc1C(=O)Nc1ccc(C2=NCCN2)cc1] | NCI-B07 |
| 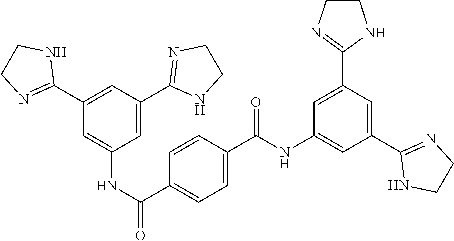 [SMILES: O=C(Nc1cc(C2=NCCN2)cc(C2=NCCN2)c1)c1ccc(C(=O)Nc2cc(C3=NCCN3)cc(C3=NCCN3)c2)cc1] | 9 (NCI-B09) |
| 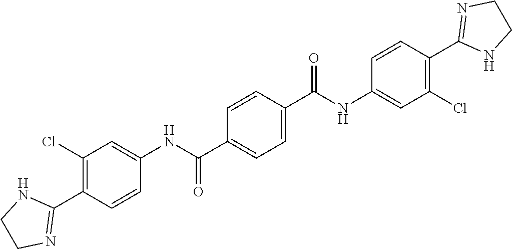 [SMILES: O=C(Nc1ccc(C2=NCCN2)c(Cl)c1)c1ccc(C(=O)Nc2ccc(C3=NCCN3)c(Cl)c2)cc1] | NCI-B13 |
| 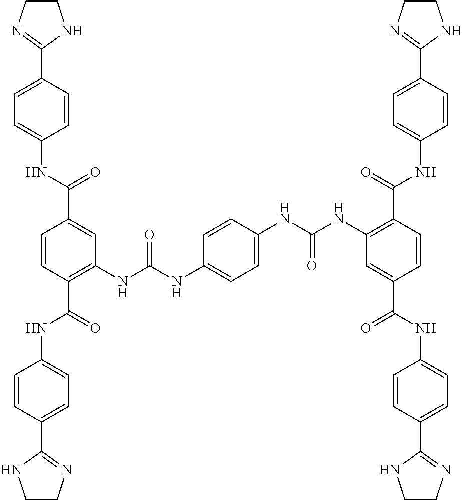 [SMILES: O=C(Nc1ccc(NC(=O)Nc2cc(C(=O)Nc3ccc(C4=NCCN4)cc3)ccc2C(=O)Nc2ccc(C3=NCCN3)cc2)cc1)Nc1cc(C(=O)Nc2ccc(C3=NCCN3)cc2)ccc1C(=O)Nc1ccc(C2=NCCN2)cc1] | NCI-B15 |
| 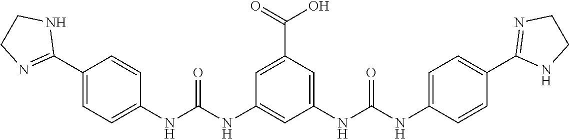 [SMILES: O=C(Nc1ccc(C2=NCCN2)cc1)Nc1cc(NC(=O)Nc2ccc(C3=NCCN3)cc2)cc(C(=O)O)c1] | NCI-B16 |
| 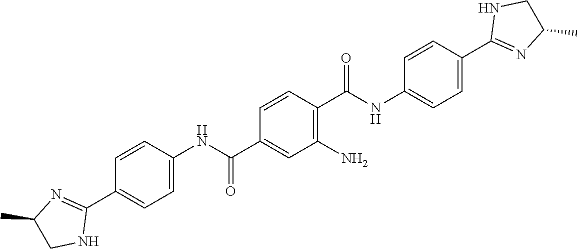 | NCI-B18 |
| 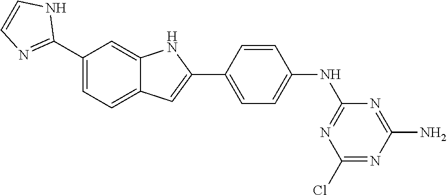 [SMILES: Nc1nc(Cl)nc(Nc2ccc(-c3cc4ccc(-c5ncc[nH]5)cc4[nH]3)cc2)n1] | NCI-B20 |
| 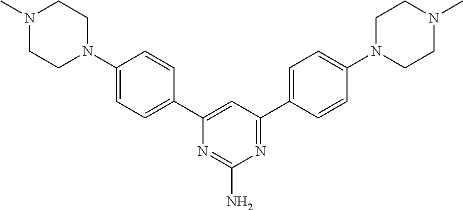 | TT76 |
| 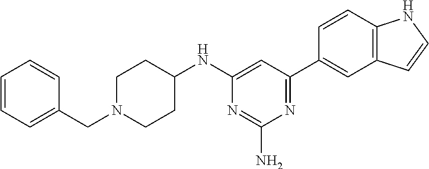 [SMILES: Nc1nc(NC2CCN(Cc3ccccc3)CC2)cc(-c2ccc3[nH]ccc3c2)n1] | TT81 |
| 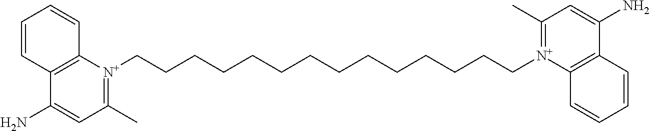 [SMILES: Cc1cc(N)c2ccccc2[n+]1CCCCCCCCCCCCCC[n+]1c(C)cc(N)c2ccccc21] | LP02 |
| 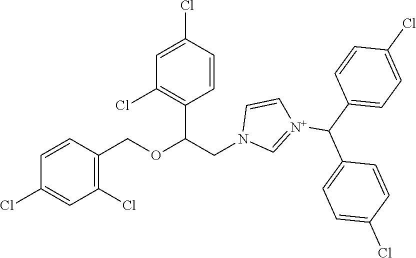 [SMILES: Clc1ccc(C(c2ccc(Cl)cc2)[n+]2ccn(CC(OCc3ccc(Cl)cc3Cl)c3ccc(Cl)cc3Cl)c2)cc1] | LP07 |
| 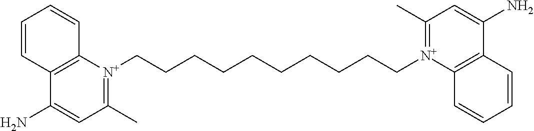 [SMILES: Cc1cc(N)c2ccccc2[n+]1CCCCCCCCCC[n+]1c(C)cc(N)c2ccccc21] | LP08 |
| 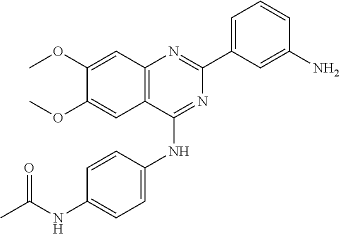 [SMILES: COc1cc2nc(-c3cccc(N)c3)nc(Nc3ccc(NC(C)=O)cc3)c2cc1OC] | S2-O22 |
| 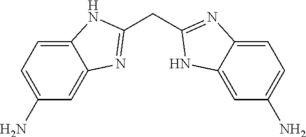 [SMILES: Nc1ccc2[nH]c(Cc3nc4ccc(N)cc4[nH]3)nc2c1] | S2-L3 |
| 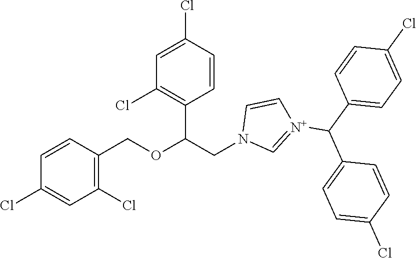 [SMILES: Clc1ccc(C(c2ccc(Cl)cc2)[n+]2ccn(CC(OCc3ccc(Cl)cc3Cl)c3ccc(Cl)cc3Cl)c2)cc1] | 1a-4 |
| 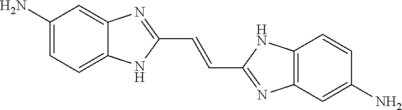 [SMILES: Nc1ccc2[nH]c(/C=C/c3nc4cc(N)ccc4[nH]3)nc2c1] | 1a-5 |
| 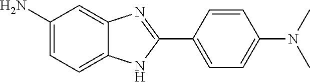 [SMILES: CN(C)c1ccc(-c2nc3cc(N)ccc3[nH]2)cc1] | 1a-8 |
| 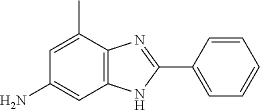 [SMILES: Cc1cc(N)cc2[nH]c(-c3ccccc3)nc12] | H1-41 |
| 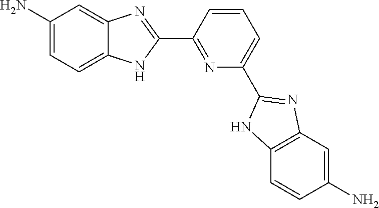 [SMILES: Nc1ccc2[nH]c(-c3cccc(-c4nc5cc(N)ccc5[nH]4)n3)nc2c1] | H1-100 |
| 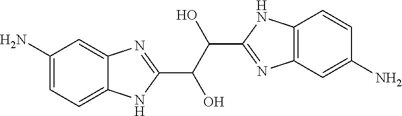 [SMILES: Nc1ccc2[nH]c(C(O)C(O)c3nc4cc(N)ccc4[nH]3)nc2c1] | H1-180 |
| 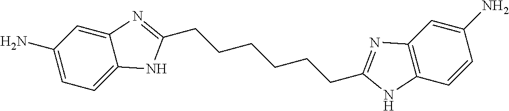 [SMILES: Nc1ccc2[nH]c(CCCCCCc3nc4cc(N)ccc4[nH]3)nc2c1] | H1-219 |
| 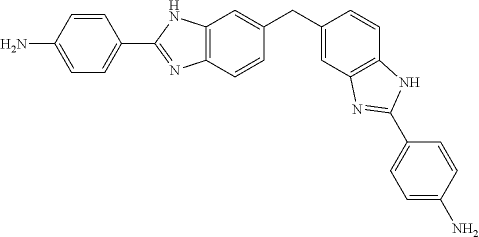 [SMILES: Nc1ccc(-c2nc3cc(Cc4ccc5nc(-c6ccc(N)cc6)[nH]c5c4)ccc3[nH]2)cc1] | H1-236 |
| 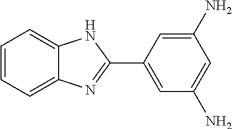 [SMILES: Nc1cc(N)cc(-c2nc3ccccc3[nH]2)c1] | H1-239 |
| 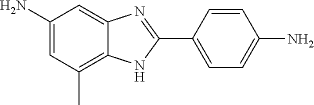 [SMILES: Cc1cc(N)cc2nc(-c3ccc(N)cc3)[nH]c12] | H1-247 |
| 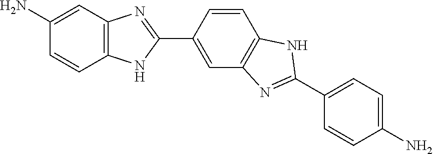 [SMILES: Nc1ccc(-c2nc3cc(-c4nc5cc(N)ccc5[nH]4)ccc3[nH]2)cc1] | H1-268 |
| 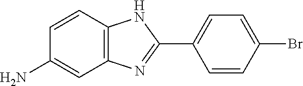 [SMILES: Nc1ccc2[nH]c(-c3ccc(Br)cc3)nc2c1] | H1-G22 |
| 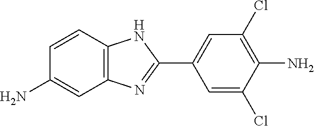 [SMILES: Nc1ccc2[nH]c(-c3cc(Cl)c(N)c(Cl)c3)nc2c1] | H1-M12 |
| 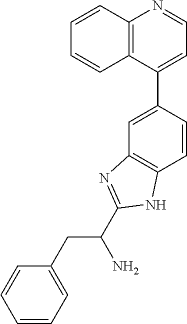 [SMILES: NC(Cc1ccccc1)c1nc2cc(-c3ccnc4ccccc34)ccc2[nH]1] | PL270-F6 |
| 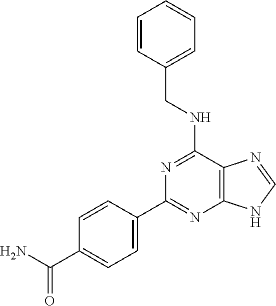 [SMILES: NC(=O)c1ccc(-c2nc(NCc3ccccc3)c3nc[nH]c3n2)cc1] | PL270-H13 |
| 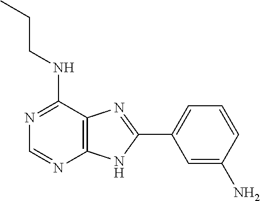 [SMILES: CCCNc1ncnc2[nH]c(-c3cccc(N)c3)nc12] | PL270-H20 |
| 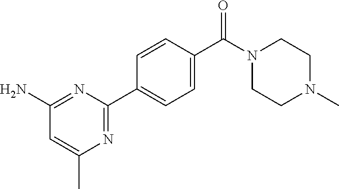 [SMILES: Cc1cc(N)nc(-c2ccc(C(=O)N3CCN(C)CC3)cc2)n1] | BV-C4 |
| 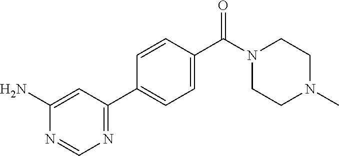 [SMILES: CN1CCN(C(=O)c2ccc(-c3cc(N)ncn3)cc2)CC1] | BV-C5 |
| 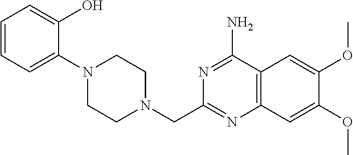 [SMILES: COc1cc2nc(CN3CCN(c4ccccc4O)CC3)nc(N)c2cc1OC] | LD-104 |
| 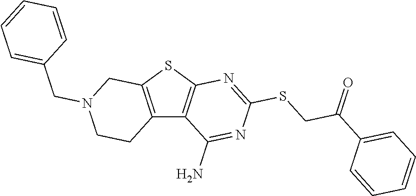 [SMILES: Nc1nc(SCC(=O)c2ccccc2)nc2sc3c(c12)CCN(Cc1ccccc1)C3] | LD-381 |
| 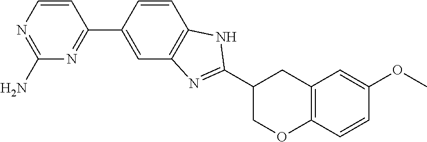 [SMILES: COc1ccc2c(c1)CC(c1nc3cc(-c4ccnc(N)n4)ccc3[nH]1)CO2] | LD-565 |
| 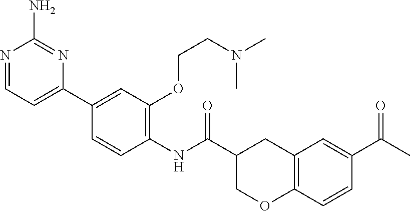 [SMILES: CC(=O)c1ccc2c(c1)CC(C(=O)Nc1ccc(-c3ccnc(N)n3)cc1OCCN(C)C)CO2] | LD-592 |
| 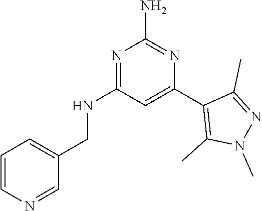 [SMILES: Cc1nn(C)c(C)c1-c1cc(NCc2cccnc2)nc(N)n1] | LD-611 |
| 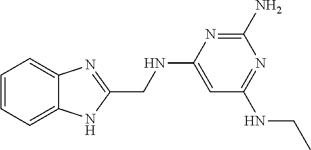 [SMILES: CCNc1cc(NCc2nc3ccccc3[nH]2)nc(N)n1] | LD-633 |
| 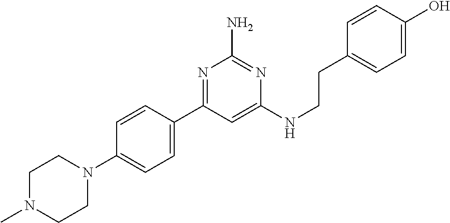 [SMILES: CN1CCN(c2ccc(-c3cc(NCCc4ccc(O)cc4)nc(N)n3)cc2)CC1] | LD-638 |
| 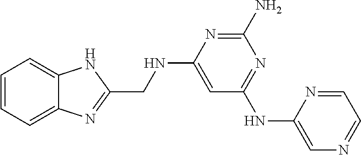 [SMILES: Nc1nc(NCc2nc3ccccc3[nH]2)cc(Nc2cnccn2)n1] | LD-641 |
| 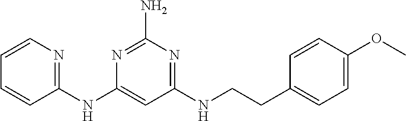 [SMILES: COc1ccc(CCNc2cc(Nc3ccccn3)nc(N)n2)cc1] | LD-643 |
| 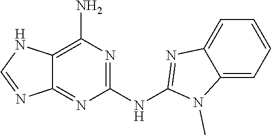 [SMILES: Cn1c(Nc2nc(N)c3[nH]cnc3n2)nc2ccccc21] | LD-645 |
| 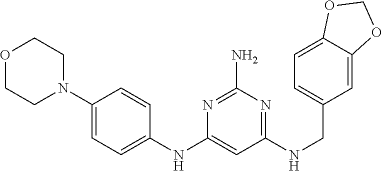 [SMILES: Nc1nc(NCc2ccc3c(c2)OCO3)cc(Nc2ccc(N3CCOCC3)cc2)n1] | LD-648 |
| 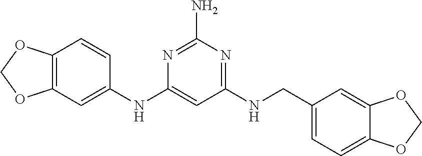 [SMILES: Nc1nc(NCc2ccc3c(c2)OCO3)cc(Nc2ccc3c(c2)OCO3)n1] | LD-657 |
| 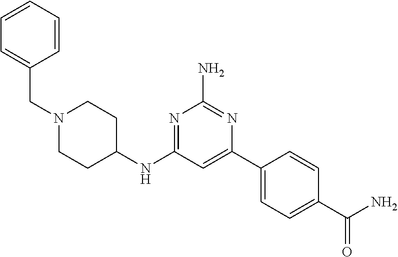 [SMILES: NC(=O)c1ccc(-c2cc(NC3CCN(Cc4ccccc4)CC3)nc(N)n2)cc1] | LD-658 |
| 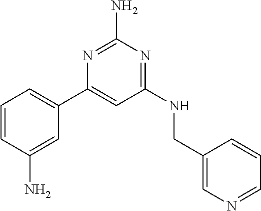 [SMILES: Nc1cccc(-c2cc(NCc3cccnc3)nc(N)n2)c1] | LD-665 |
| 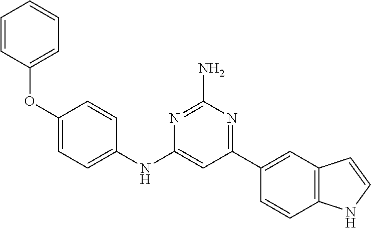 [SMILES: Nc1nc(Nc2ccc(Oc3ccccc3)cc2)cc(-c2ccc3[nH]ccc3c2)n1] | LD-666 |
| 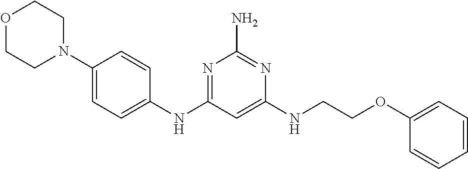 [SMILES: Nc1nc(NCCOc2ccccc2)cc(Nc2ccc(N3CCOCC3)cc2)n1] | LD-702 |
| 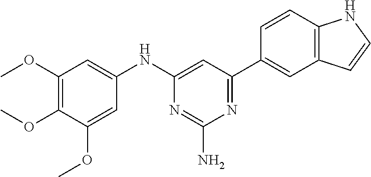 [SMILES: COc1cc(Nc2cc(-c3ccc4[nH]ccc4c3)nc(N)n2)cc(OC)c1OC] | LD-704 |
| 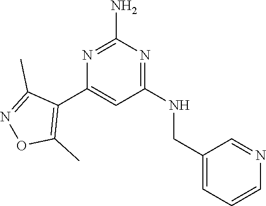 [SMILES: Cc1noc(C)c1-c1cc(NCc2cccnc2)nc(N)n1] | LD-714 |
| 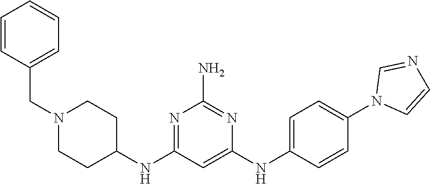 [SMILES: Nc1nc(Nc2ccc(-n3ccnc3)cc2)cc(NC2CCN(Cc3ccccc3)CC2)n1] | LD-717 |
| 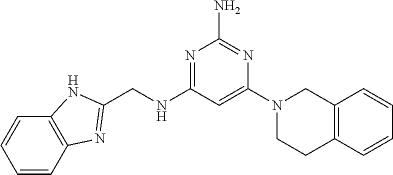 [SMILES: Nc1nc(NCc2nc3ccccc3[nH]2)cc(N2CCc3ccccc3C2)n1] | LD-720 |
| 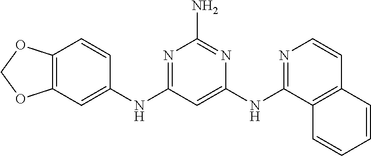 [SMILES: Nc1nc(Nc2ccc3c(c2)OCO3)cc(Nc2nccc3ccccc23)n1] | LD-1003 |
| 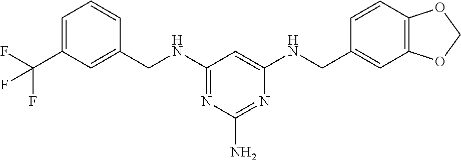 [SMILES: Nc1nc(NCc2cccc(C(F)(F)F)c2)cc(NCc2ccc3c(c2)OCO3)n1] | LD-1005 |
| 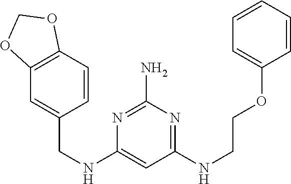 [SMILES: Nc1nc(NCCOc2ccccc2)cc(NCc2ccc3c(c2)OCO3)n1] | LD-1009 |
| 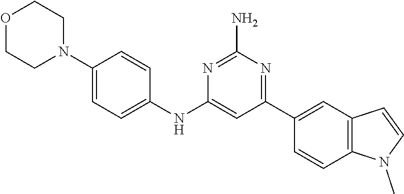 [SMILES: Cn1ccc2cc(-c3cc(Nc4ccc(N5CCOCC5)cc4)nc(N)n3)ccc21] | LD-1018 |
| 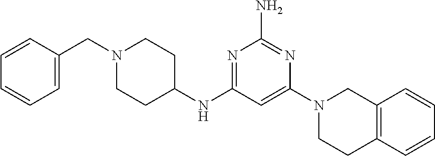 [SMILES: Nc1nc(NC2CCN(Cc3ccccc3)CC2)cc(N2CCc3ccccc3C2)n1] | LD-1025 |
| 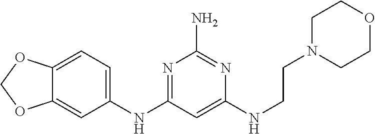 [SMILES: Nc1nc(NCCN2CCOCC2)cc(Nc2ccc3c(c2)OCO3)n1] | LD-1033 |
| 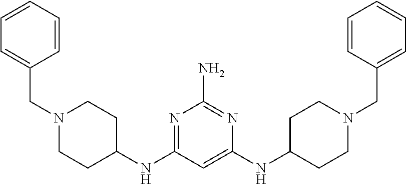 [SMILES: Nc1nc(NC2CCN(Cc3ccccc3)CC2)cc(NC2CCN(Cc3ccccc3)CC2)n1] | LD-1034 |
| 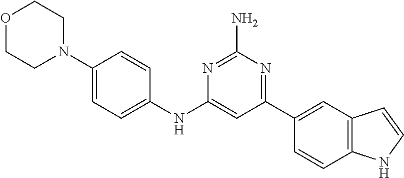 [SMILES: Nc1nc(Nc2ccc(N3CCOCC3)cc2)cc(-c2ccc3[nH]ccc3c2)n1] | LD-1035 |
| 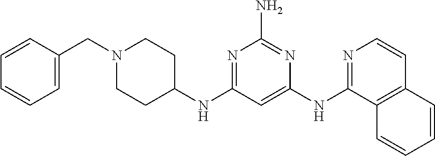 [SMILES: Nc1nc(Nc2nccc3ccccc23)cc(NC2CCN(Cc3ccccc3)CC2)n1] | LD-1037 |
| 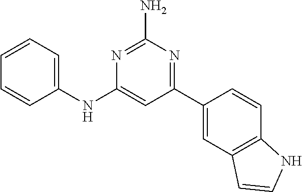 [SMILES: Nc1nc(Nc2ccccc2)cc(-c2ccc3[nH]ccc3c2)n1] | LD-1040 |
| 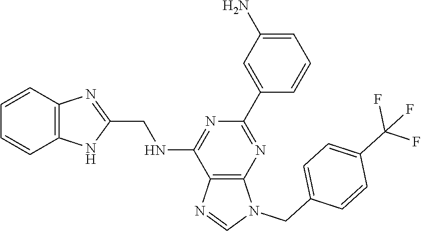 [SMILES: Nc1cccc(-c2nc(NCc3nc4ccccc4[nH]3)c3ncn(Cc4ccc(C(F)(F)F)cc4)c3n2)c1] | LD-1060 |
| 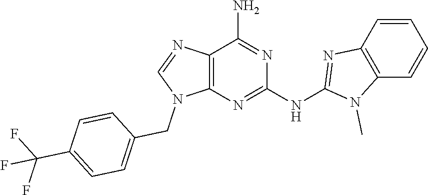 [SMILES: Cn1c(Nc2nc(N)c3ncn(Cc4ccc(C(F)(F)F)cc4)c3n2)nc2ccccc21] | LD-1063 |
| 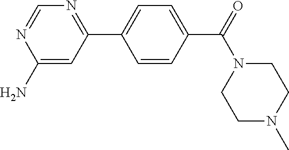 [SMILES: CN1CCN(C(=O)c2ccc(-c3cc(N)ncn3)cc2)CC1] | LD-1076 |
| 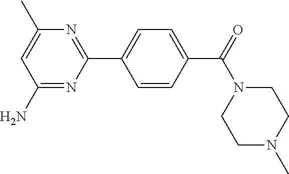 [SMILES: Cc1cc(N)nc(-c2ccc(C(=O)N3CCN(C)CC3)cc2)n1] | LD-1077 |
| 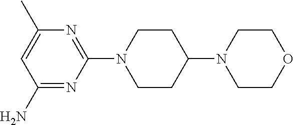 [SMILES: Cc1cc(N)nc(N2CCC(N3CCOCC3)CC2)n1] | LD-1086 |
| 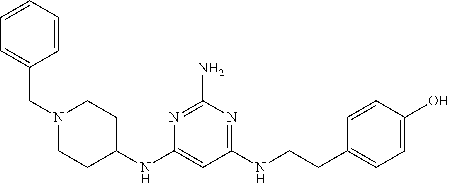 [SMILES: Nc1nc(NCCc2ccc(O)cc2)cc(NC2CCN(Cc3ccccc3)CC2)n1] | LD-1257 |
| 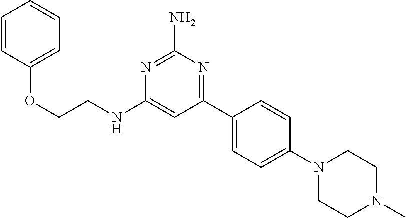 [SMILES: CN1CCN(c2ccc(-c3cc(NCCOc4ccccc4)nc(N)n3)cc2)CC1] | LD-1287 |
| 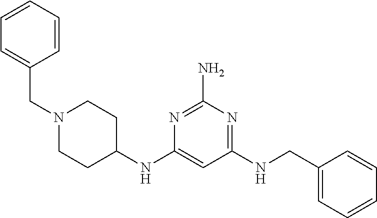 [SMILES: Nc1nc(NCc2ccccc2)cc(NC2CCN(Cc3ccccc3)CC2)n1] | LD-1288 |
| 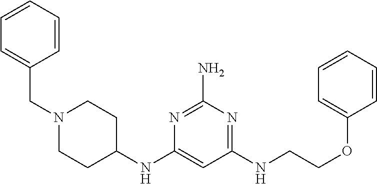 [SMILES: Nc1nc(NCCOc2ccccc2)cc(NC2CCN(Cc3ccccc3)CC2)n1] | LD-1310 |
| 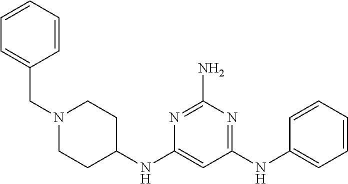 [SMILES: Nc1nc(Nc2ccccc2)cc(NC2CCN(Cc3ccccc3)CC2)n1] | LD-1313 |
| 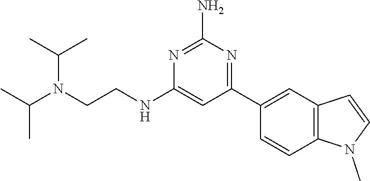 [SMILES: CC(C)N(CCNc1cc(-c2ccc3c(ccn3C)c2)nc(N)n1)C(C)C] | LD-1360 |
| 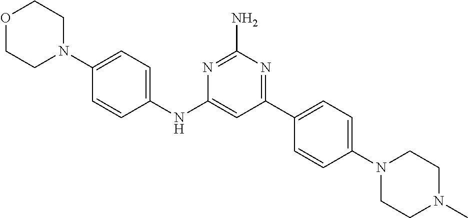 [SMILES: CN1CCN(c2ccc(-c3cc(Nc4ccc(N5CCOCC5)cc4)nc(N)n3)cc2)CC1] | LD-1361 |
| 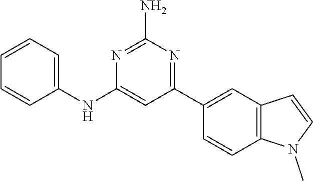 [SMILES: Cn1ccc2cc(-c3cc(Nc4ccccc4)nc(N)n3)ccc21] | LD-1362 |
| 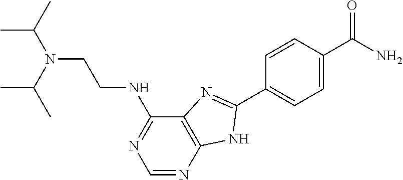 [SMILES: CC(C)N(CCNc1ncnc2[nH]c(-c3ccc(C(N)=O)cc3)nc12)C(C)C] | LD-1364 |
| 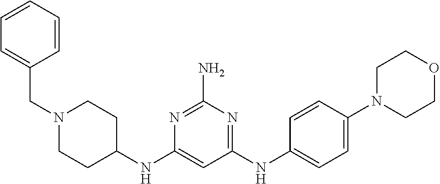 [SMILES: Nc1nc(Nc2ccc(N3CCOCC3)cc2)cc(NC2CCN(Cc3ccccc3)CC2)n1] | LD-1379 |
| 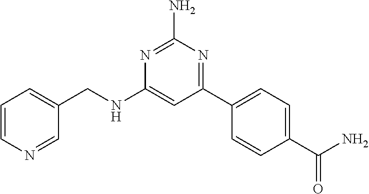 [SMILES: NC(=O)c1ccc(-c2cc(NCc3cccnc3)nc(N)n2)cc1] | LD-1380 |
| 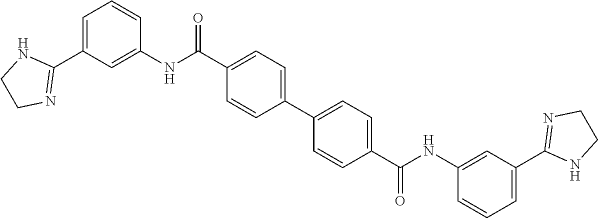 [SMILES: O=C(Nc1cccc(C2=NCCN2)c1)c1ccc(-c2ccc(C(=O)Nc3cccc(C4=NCCN4)c3)cc2)cc1] | 10 (NCI-B4) |

TABLE 4
List of primers used for RT-qPCR and
associated experiments

| Primer Name | Sequence 5′ - 3′ |
|---|---|
| FGFR1 | GCACATCCAGTGGCTAAAGCAC (SEQ ID NO: 10) |
| FGFR1 [symbol] | AGCACCTCATCTCTTTGTCGG (SEQ ID NO: 11) |
| JAK3 | AGTGACCCTCACTTCCTGT (SEQ ID NO: 12) |
| JAK3 [symbol] | GGCTGAACCAAGGATGATGTGG (SEQ ID NO: 13) |
| PAK1 | GTGAAGGCTGTGTCTGAGACTC (SEQ ID NO: 14) |
| PAK1 [symbol] | GGAAGTGGTTCAATCACAGACCG (SEQ ID NO: 15) |
| IRAK4 | ATGCCACCTGACTCCTCAAGT (SEQ ID NO: 16) |
| IRAK4 [symbol] | CCACCAACAGAAATGGGTCGTTC (SEQ ID NO: 17) |
| P38α (MAPK) | GAGCGTTACCAAACCTGTCTC (SEQ ID NO: 18) |
| P38α [symbol] | AGTAACCGCAGTTCTCTGTAGGT (SEQ ID NO: 19) |
| AMPKA1 | AGGAAGAATCCTGTGACAAGCAC (SEQ ID NO: 20) |
| AMPKA1 [symbol] | CCGATCTCTGTGGAGTAGCAGT (SEQ ID NO: 21) |
| AMPKB1 | CTCCAGGTCATCCTGAACAAGG (SEQ ID NO: 22) |
| AMPKB1 [symbol] | ACAGCGCGTATAGGTGGTTCAG (SEQ ID NO: 23) |
| AMPKG1 | AGAGGTTCTGCCCGTCTTCCTT (SEQ ID NO: 24) |
| AMPKG1 [symbol] | TTCGTCCTCGAACTCCAGCTT (SEQ ID NO: 25) |
| CAMK4 | GTTCTTCTTCGCCTCTCACATCC (SEQ ID NO: 26) |
| CAMK4 [symbol] | CTGTGACGAGTTCTAGGACCAG (SEQ ID NO: 27) |
| CHK1 | GTGTCAGAGTCTCCCAGTGGAT (SEQ ID NO: 28) |
| CHK1 [symbol] | GTTCTGGCTGAGAACTGGAGTAC (SEQ ID NO: 29) |
| AKT | TGGACTACCTGCACTCGGAGAA (SEQ ID NO: 30) |
| AKT [symbol] | GTGCCGCAAAAGGTCTTCATGG (SEQ ID NO: 31) |
| ROCK1 | GAAACAGTGTTCCATGCTAGACG (SEQ ID NO: 32) |
| ROCK1 [symbol] | GCCGCTTATTTGATTCCTGCTCC (SEQ ID NO: 33) |
| β-Actin | TGTGATGGTGGGAATGGGTCAGAA (SEQ ID NO: 34) |
| β-Actin [symbol] | TGTGGTGCCAGATCTTCTCCATGT (SEQ ID NO: 35) |
| HCV-3′-UTR-SL1, II, | TGGTGGCTCCATCTTAGCCC (SEQ ID NO: 36) |
| III | |
| HCV-3′-UTR-SL1, II, | CCTGCAGGTCGACTCTAG (SEQ ID NO: 37) |
| III [symbol] | |
| HCV-3′UTR Ctrl2 | CATCGTGGTGTCACGC (SEQ ID NO: 38) |
| HCV-3′UTR Ctrl2 [symbol] | ATCATGTAACTCGCCTT (SEQ ID NO: 39) |
| SL I ASO | mA*mC*mG*G*C*A*C*T*C*T*C*T*G*mC*mA*mG |
| | (SEQ ID NO: 40) |
| Control ASO | mG*mG*mG*A*A*C*C*G*G*A*G*C*T*mG*mA* |
| | (SEQ ID NO: 41) |

[symbol]

= Reverse primer,
m= 2′-O-methyl modified deoxynucleotide
*= Phosphorothioate backbone.
All oligonucleotides were used without further purification.

### Examples

### Methods:

Oligonucleotides. All DNA oligonucleotides were purchased from Integrated DNA Technologies, Inc. (IDT) and used without further purification. RNA oligonucleotide competitors were purchased from Dharmacon and de-protected according to the manufacturer's standard procedure. All oligonucleotide solutions were prepared with NANOpure water. The RNA motif libraries were synthesized by in vitro transcription from the corresponding DNA template that was custom-mixed at the randomized positions to ensure equivalent representation of all four nucleotides.
Compounds. All compounds were either obtained from the National Cancer Institute (NCI), The Scripps Research Institute (TSRI), or the National Institutes of Health (NIH).
Calculation of Lipinski's Parameters and Chemoinformatic Analysis. JChem for Excel software was used to calculate c Log P, c Log D, molecular weight, H-bond donor, H-bond acceptors, rotatable bonds and shape Tanimoto values (JChem for Excel 5.11.4.886, 2012, ChemAxon (http://www.Chemaxon.com)). Chemoinformatic analysis of chemical substructures were generated by JChem (JChem 5.8.0, 2012, ChemAxon, http://www.chemaxon.com) and by NCGC Automatic R-group analysis program (Tripod Development; http://tripod.nih.gov/?p=46)¹³.
Construction of Small Molecule Microarrays. Microarrays were constructed as previously described¹¹.
RNA Selection. RNA libraries were radioactively labeled at the 5′-end as previously described using 1 mL of [γ-³²P1 ATP (3000 Ci/mol; PerkinElmer)¹⁰.
Reverse Transcription-Polymerase Chain Reaction (RT-PCR) Amplification. Bound RNAs were excised from agarose microarrays and incubated in 20 μL, of 1×RQ DNase I Buffer containing 2 units RQ1 RNase-free DNase (Promega) at 37° C. for 2 h. The sample was supplemented with 2 μL of 10×DNase Stop Solution (Promega) and incubated at 65° C. for 10 min to inactivate the DNase. This solution was used for reverse transcription-polymerase chain reaction (RT-PCR) amplification as previously describe¹⁴. Aliquots of the RT-PCR reactions were checked every five cycles starting at cycle 25 on a 15% polyacrylamide gel stained with ethidium bromide or SYBR Gold (Life Technologies).
Reverse Transcription and PCR Amplification to Install Barcodes for RNA-seq. Samples were prepared for RNA-seq analysis as previously described¹². After purification using a native 8% or 12.5% polyacrylamide gel, the desired product was re-amplified by using manufacturer provided primer sets and the following cycling conditions: 94° C. for 30 s, 60° C. for 30 s and 72° C. for 30 s.
RNA-seq. RNA-seq was completed at Next Generation Sequencing Core at The Scripps Research Institute (La Jolla, Calif.) or the Scripps Florida Genomics Core.
Identification of Privileged RNA Motifs. Privileged RNA motifs were identified by High Throughput Structure-Activity Relationships Through Sequencing (HiT-StARTS)¹². HiT-StARTS uses a pooled population comparison (Z_{obs}) to account for biases in transcription and RT-PCR. Z_{obs} is calculated for each sequence per equations 1 and 2)¹⁵:

$$\begin{matrix}ϕ = \frac{{n}_{1} {p}_{1} + {n}_{2} {p}_{2}}{{n}_{1} + {n}_{2}} & ( equation 1 ) \\ {Z}_{obs} = \frac{{p}_{1} - {p}_{2}}{\sqrt{ϕ ( 1 - ϕ ) ( \frac{1}{{n}_{1}} + \frac{1}{{n}_{2}} )}} & ( equation 2 )\end{matrix}$$

where n₁ is the size of Population 1 (number of sequencing reads for a given RNA from RNA-seq analysis of the selected library), n₂ is the size of Population 2 (number of sequencing reads for the same RNA from RNA-seq analysis of the starting library), p₁ is the observed proportion of Population 1 (the number of reads for a given RNA from selected library/total number of reads), and p₂ is the observed proportion for Population 2 (the number of reads for the same RNA in the starting library/total number of reads).
Generation of Logos and DiffLogos: Z_{obs} corresponding to the highest (enriched) 0.5% and lowest (discriminated against) 0.5% scores were analyzed to generate consensus sequences, or Logos. The resulting list of sequences were converted to position weight matrix (PWM) lists for each compound. “Enriched” and “discriminated” sequences were kept separate using JMP®, Version <13.2.1>. SAS Institute Inc., Cary, N.C., 1989-2007. The R package Difflogo,¹⁶ which is part of Bioconductor, was utilized to create the sequence logos from PWM lists for each compound and visually compare the differences between them.
Binding Affinity Measurements. Dissociation constants were determined using an in solution, fluorescence-based assay. Briefly, 100 nM of RNA labeled with 5′ fluorescein was folded in 1× Assay Buffer (20 mM HEPES, pH 7.5, 150 mM NaCl, 5 mM KCl, and 40 μg/mL BSA) by heating at 60° C. for 5 min and slowly cooling to room temperature, after which MgCl₂ was added to a final concentration of 1 mM. Serial dilutions (1:2) of the small molecule were then completed in 1× Assay Buffer supplemented with 1 mM MgCl₂. The solutions were incubated for 30 min at room temperature, transferred to a 96-well plate, and fluorescence intensity measured on a BioTek-FLx800 plate reader. The change in fluorescence intensity as a function of small molecule concentration was fit to a one site binding model per equation 3:

$$F l = \frac{{B}_{max} \times {X}^{H}}{{K}_{d} + {X}^{H}} \qquad ( equation 3 )$$

where Fl is the fluorescence intensity, Bₘₐₓ is the maximum specific binding, X is the concentration of the small molecule, H is the Hill slope, and K_{d} is the dissociation constant.
Effect of compounds on HCV replication. Hepatitis C virus stable sub-genomic replicon cells [SGR-Rluc-Neo-(NS3-5B)-JFH1-2a with luciferase reporter] were plated in 24-well plates (5×10⁴ cells/well). Approximately 24 h post cell plating, the compound of interest was added. DMSO (vehicle) or 2′-C-methyladenosine triphosphate¹² (10 mM concentration) were included as controls. After 72 h, cells were washed twice with 1×PBS followed by addition of 400 mL of 1× luciferase lysis buffer. A 20 mL aliquot of cell lysate was used to measure luciferase activity per the manufacturer's instructions (Promega).
Cytotoxicity assays. Huh 7.5 cells (same cells used for the development of HCV stable replicon cells) were plated in 24-well plate (5×10⁴ cells/well). After 24 h, the compound of interest was added and incubated with the cells for 72 h. Cell viability was measured using a CellTiter-Glo (Promega) or WST-1 per manufacturer's instructions.
Oligonucleotide profiling of target engagement in vitro. WT-SLI and BP-SLI RNAs were 5′-end labeled as previously described. The RNA of interest (100 nM) was in 1× RNase H Reaction Buffer (New England BioLabs) by heating at 60° C. for 5 min and slowly cooling to room temperature. The RNA was incubated with compound (0.1, 1, 5 and 10 μM) for 15 min at room temperature. Then, the corresponding antisense oligonucleotide (Table S4) was added to a final concentration of 1 μM and incubated for another 15 min before addition of RNase H to a final concentration of 0.05 U/μL. The samples were incubated at 37° C. or 30 min and the resulting fragments were separated on a denaturing 15% polyacrylamide gel.
Oligonucleotide profiling of target engagement by RT-qPCR. Hepatitis C virus stable sub-genomic replicon cells were plated in 100 mm dishes and grown to ˜80% confluency. The cells were batch transfected with antisense oligonucleotides (50 nM) targeting the SLI site or a control site using Lipofectamine 2000 (Life Technologies) per the manufacturer's protocol. After removing the transfection cocktail, the cells were allowed to recover in growth medium for 4 h before seeding into 24-well plates (300,000 cells/well). Cells were allowed to adhere for 2 h before treatment with 8 for 24 h. Total RNA was then extracted using Zymo Quick-RNA mini-prep kit per the manufacturer's protocol. RT was carried out with qScript reverse transcriptase (Quantabio). qPCR was completed using Power SYBR PCR Master Mix and an Applied Biosystems 7900 HT cycler with the following cycling conditions: 95° C., 30 s; 55° C., 30 s; 72° C., 30 s. Primers can be found in Table 4. Many RNAs cause disease, however RNA is rarely exploited as a small molecule drug target. A programmatic focus is to define privileged RNA motif-small molecule interactions to enable the rational design of compounds that modulate RNA biology starting from only sequence. A massive, library-versus-library screen was completed that probed over 50 million binding events between RNA motifs and small molecules. The resulting data provide a rich encyclopedia of small molecule-RNA recognition patterns, defining chemotypes and RNA motifs that confer selective, avid binding. The resulting interaction maps were mined against the entire viral genome of hepatitis C virus (HCV). A small molecule was identified that avidly bound RNA motifs present in the HCV 3′ untranslated region and inhibited viral replication while having no effect on host cells. Collectively, this study represents the first whole genome pattern recognition between small molecules and RNA folds.
Inhibition of kinases. Compounds 7-10 and Staurosporine, a pan kinase inhibitor, were tested for in vitro inhibition of a panel of 22 kinases (20 μM, 24 and 72 h). As expected, Staurosporine is a global kinase inhibitor, on average inhibiting 84±3% of kinase activity. Compounds 7 and 8 each affected seven kinases modestly (average percent inhibition of 16±3% and 14±3%, respectively), with CAMK4 the most significantly inhibited. No kinases were significantly inhibited by 9 or 10. To determine if anti-HCV activity might be traced to inhibition of kinases, we measured the mRNA expression levels of the seven affected kinases in the Huh-7.5 replicon cell line by RT-qPCR. Interestingly, CAMK4, which is the most significantly inhibited by 7 and 8 in vitro is not expressed in Huh-7.5 cells, therefore making it an unlikely cellular target of our lead compounds.
To gain insight into if the kinases inhibited by 7 and 8 in vitro might give rise to anti-HCV activity, the activity of known kinase inhibitors was studied in the replicon assay (20 μM; Figure S7C). Inhibitors of JAK3, CAMK, p38a (MAPK), and ROCK1 have no activity in the assay while inhibitors of AKT and CHK1 inhibit ˜90% and ˜50% of HCV replication. Notably, CHK1 is inhibited by 8 but not 7 in vitro. AKT is a serine/threonine kinase that controls cell proliferation. Thus, an argument could be made that 7 and 8's anti-HCV activity in the replicon assay could be traced to inhibition of AKT and hence proliferation and decreased replication. However, neither 7 or 8 significantly affected cell viability. In addition, 96 known kinase inhibitors (200 nM) were profiled for anti-HCV activity. After 24 h (incubation time for our lead compounds), 12 compounds showed >20% inhibition of HCV replication. Collectively, these results suggest that the modes of action for 7 and 8 are not due to general inhibition of cellular kinases.

Thus, several approaches including kinase profiling, structure-activity relationship for RNA binding, and competitive oligonucleotide profiling collectively support that the compounds modulate HCV by directly targeting the viral RNA.

Summary & Outlook. Herein, diverse, drug-like small molecules were identified with preferences for particular RNA motifs. It was previously shown that a fundamental understanding of the motifs preferred by small molecules can inform design of selective modulators of RNA (dys)function. Indeed, this “bottom-up” approach has afforded chemical probes and preclinical modalities against expanded repeating RNAs that cause neurological and neuromuscular disease^{59, 61-64} and oncogenic microRNAs^{8, 65}. These studies have further established an encyclopedia of binding landscapes, in particular for drug-like small molecules, that will aid the emerging field of RNA drug design and discovery.

Further, an inhibitor was designed with similar potency that binds a viral RNA, rather than an enzyme. Indeed, these studies are the first example of using whole genome-based rational design to deliver small molecules that target a virus. As viral populations and their threats to human health ebb and flow with each season, the ability to rapidly identify antivirals by using sequence-based design could enable a paradigm shift in how viruses are targeted including drug-resistant populations. Small molecules that target RNA motifs conserved across related viruses might have broad spectrum anti-viral activity, i.e., activity against multiple viruses. Such approaches should also be applicable to DNA viruses by targeting viral RNA intermediates/transcripts.

What is claimed is:

1. A method comprising
contacting a library of RNA sequences, a complementary antisense oligonucleotide, RNase H, and a small molecule candidate RNA-binding compound and determining cleavage of the RNA sequences in the presence of the compound (“presence cleavage”); and
contacting the library of RNA sequences, the complementary antisense oligonucleotide, and RNase H in the absence of the small molecule candidate RNA-binding compound and determining cleavage of the RNA sequences in the absence of the compound (“absence cleavage”);
wherein when cleavage is inhibited (e.g., presence cleavage is lower than absence cleavage), the small molecule candidate RNA-binding compound binds to the RNA sequence.

2. The method of claim 1 wherein the RNA sequence library comprises a transcriptome.

3. The method of claim 2 wherein the transcriptome is viral.

4. The method of claim 2 wherein the transcriptome is mammalian.

5. The method of claim 2 wherein the transcriptome is bacterial.

6. The method of claim 1 wherein the RNA sequence library comprises one or more of synthetic, semi-synthetic, or natural RNA.

7. The method of claim 1 wherein the RNA sequence library comprises the genome of an RNA virus.

8. The method of claim 1 carried out in vitro.

9. The method of claim 1 carried out in living cells.

10. The method of claim 9 wherein the cells are virally- or bacterially-infected cells.

11. The method of claim 1 wherein a set of complementary antisense oligonucleotides and a set of small molecule candidate RNA-binding compounds are assayed in a 2-dimensional parallel array.
